(19) United States
(12) Patent Application Publication
(10) Pub. No.: US 2026/0278699 A1
(43) Pub. Date: Sep. 17, 2026

(54) ORDERING AND MANUFACTURING SYSTEM FOR 3D-PRINTED BOLUS MEDICAL DEVICES

(71) Applicants: Adam Wentworth, Rochester, MN (US); Scott Brewer, Castle Rock, CO (US); Eva Mueller, Winston-Salem, NC (US); Brian Overshiner, Winston-Salem, NC (US); Steven Nelson, San Jose, CA (US); Giovanni Bisaccia, San Jose, CA (US); Ryan Hess, Cary, NC (US)

(72) Inventors: Adam Wentworth, Rochester, MN (US); Scott Brewer, Castle Rock, CO (US); Eva Mueller, Winston-Salem, NC (US); Brian Overshiner, Winston-Salem, NC (US); Steven Nelson, San Jose, CA (US); Giovanni Bisaccia, San Jose, CA (US); Ryan Hess, Cary, NC (US)

(73) Assignee: Ricoh Company, Ltd., Toyko (JP)

(21) Appl. No.: 19/077,637

(22) Filed: Mar. 12, 2025

(51) Int. Cl. G06Q 50/04; B33Y 50/00; G16H 40/00

(52) U.S. Cl. CPC G06Q 50/04; G16H 40/00; B33Y 50/00

(57) ABSTRACT

A computer-implemented system, method and computer program product for ordering and manufacturing 3D-printed bolus medical devices, include an order processing system configured to receive order information and patient imaging data for a bolus medical device. A bolus design system is coupled to the order processing system and includes a bolus design script device configured to generate a patient-specific bolus based on anatomical and/or treatment parameters related to the patient, and to generate a finalized bolus design. A production system is coupled to the bolus design system and configured to receive the finalized bolus design and perform 3D printing of a bolus medical device based on the finalized bolus design. A delivery module is configured to deliver the 3D-printed bolus medical device to a treatment facility.

### CROSS REFERENCE TO RELATED DOCUMENTS

The present disclosure is related to U.S. patent application Attorney Docket No. 006416US of NELSON et al., entitled “AI-ENHANCED ORDERING AND MANUFACTURING SYSTEM FOR 3D-PRINTED MEDICAL DEVICES,” filed 12 Mar. 2025, now, U.S. patent application Ser. No. 17/196,842 of HESS et al., entitled “3D PRINT PORTAL TO ASSIST IN REVISING, REVIEWING, AND APPROVING 3D PRINTABLE FILES,” filed on 9 Mar. 2021, now, U.S. patent application Ser. No. 17/688,795 of NELSON et al., entitled “PROVIDING CLEARANCE, NOTIFICATION, AND SELECTION OF USE RELATED TO CERTIFICATION OF 3D ANATOMICAL MODELS FOR PRESURGICAL PLANNING,” filed on 7 Mar. 2022, now, U.S. patent application Ser. No. 17/688,808 of NELSON et al., entitled “PRINTING 3D ANATOMICAL MODELS FOR DIAGNOSTIC AND NON-DIAGNOSTIC USES,” filed on 7 Mar. 2022, now U.S. Pat. No. 11,789,682 issued 17 Oct. 2023, U.S. patent application Ser. No. 18/608,911 of NELSON et al., entitled “SYSTEM ARCHITECTURE FOR UPLOADING IMAGE DATA AND APPROVING SEGMENTED IMAGE DATA,” filed on 18 Mar. 2024, now pending, and U.S. patent application Ser. No. 18/608,919 of NELSON et al., entitled “LIFECYCLE MANAGEMENT FOR PROCESSING ANATOMICAL MODELS,” filed on 18 Mar. 2024, now pending, the entire disclosures of all of which are hereby incorporated by reference herein.

### BACKGROUND OF THE DISCLOSURE

### Field of the Disclosure

The present disclosure generally relates to systems and methods for improving ordering and manufacturing of medical devices, and more particularly to systems and methods for ordering and manufacturing of 3D printed bolus medical devices, and the like.

### Discussion of the Background

In recent years, methods and systems have been developed for ordering and manufacturing 3D-printed medical devices, particularly patient-specific boluses for radiation therapy. However, these systems often rely on manual design processes, separate third-party software tools, and non-integrated workflows, leading to inefficiencies, errors, and delays. Clinicians and technicians frequently need to manually import imaging data, adjust bolus parameters, and validate design outputs, disrupting clinical workflows and increasing the risk of inaccuracies, particularly in precise radiation treatment planning.

### SUMMARY OF THE DISCLOSURE

Therefore, there is a need for a method and system that addresses the above and other problems. The above and other problems are addressed by the illustrative embodiments of the present disclosure, which provide systems and methods for the ordering and manufacturing of 3D-printed bolus medical devices. The systems and methods streamline and automate the entire process, from bolus design and validation to manufacturing and delivery. Advantageously, features such as automated bolus generation, real-time imaging data integration, script-driven design validation, and seamless workflow integration with clinical systems are included. The systems and methods minimize manual input, reduce errors, and accelerate production, making it easier for medical professionals to generate patient-specific boluses with precision and efficiency, ensuring timely delivery for radiation therapy procedures.

Accordingly, in illustrative aspects of the present disclosure there is provided a computer-implemented system, method and computer program product for ordering and manufacturing 3D-printed bolus medical devices, include an order processing system configured to receive order information and patient imaging data for a bolus medical device. A bolus design system is coupled to the order processing system and includes a bolus design script device configured to generate a patient-specific bolus based on anatomical and/or treatment parameters related to the patient, and to generate a finalized bolus design. A production system is coupled to the bolus design system and configured to receive the finalized bolus design and perform 3D printing of a bolus medical device based on the finalized bolus design. A delivery module is configured to deliver the 3D-printed bolus medical device to a treatment facility.

The order processing system includes an order information processing device coupled to a digital imaging and communications in medicine (DICOM) uploading device coupled to an order confirmation device coupled to the bolus design system.

The bolus design script device is further configured to perform at least one of part selection, dimensional evaluation, smoothing operations, Boolean subtraction, deviation analysis, and labeling.

The bolus design script device is further configured to generate a custom 3D bolus medical device, based on an imported anatomical surface scan, and based on received user input defining at least one of bolus thickness, and anatomical region selection of the custom 3D bolus medical device.

The bolus design script device is further configured to validate a preexisting 3D bolus medical device structure by performing at least one of dimensional analysis, Boolean subtraction of an anatomical model, smoothing operations, and sharp edge removal on the preexisting 3D bolus medical device structure.

The bolus design script device is further configured to generate a 3D-printed applicator medical device for brachytherapy, based on user input defining at least one of catheter diameter, catheter spacing, and offset distance from a body surface.

Still other aspects, features, and advantages of the present disclosure are readily apparent from the following detailed description, by illustrating a number of illustrative embodiments and implementations, including the best mode contemplated for carrying out the present disclosure. The present disclosure is also capable of other and different embodiments, and its several details can be modified in various respects, all without departing from the spirit and scope of the present disclosure. Accordingly, the drawings and descriptions are to be regarded as illustrative in nature, and not as restrictive.

### BRIEF DESCRIPTION OF THE DRAWINGS

The embodiments of the present disclosure are illustrated by way of example, and not by way of limitation, in the figures of the accompanying drawings and in which like reference numerals refer to similar elements and in which:

FIG. 1 shows an ordering and manufacturing workflow for 3D-printed bolus medical devices, according to the present disclosure;

FIG. 2 shows an ordering and manufacturing decision workflow system for 3D-printed bolus medical devices, according to the present disclosure;

FIG. 3 shows a bolus design script workflow for generating a patient-specific bolus in an automated and iterative manner, according to the present disclosure;

FIGS. 4-14 show a bolus design script graphical user interface (GUI) for the bolus design script workflow of FIG. 3, according to the present disclosure;

FIGS. 15-22 show a further bolus design script graphical user interface (GUI) for the bolus design script workflow of FIG. 3, according to the present disclosure;

FIGS. 23-40 show a brachytherapy applicator design script graphical user interface (GUI) that can be used with the bolus design script workflow of FIG. 3, according to the present disclosure;

FIG. 41 shows design data generated by the bolus scripts of FIGS. 4-40, according to the present disclosure;

FIGS. 42A-42C show an STL file 3D render of a scalp bolus design of FIG. 41 captured from different perspectives, according to the present disclosure; and

FIGS. 43A-43C show a 3D print of the STL file 3D render of FIGS. 42A-42C, according to the present disclosure.

DETAILED DESCRIPTION OF THE PREFERRED EMBODIMENTS

The present disclosure includes recognition that current methods for ordering and manufacturing 3D-printed bolus medical devices face several challenges, including reliance on manual design processes, the use of separate third-party software tools, fragmented workflows that disrupt clinical operations, and the like. Existing systems often require clinicians and technicians to manually import imaging data, adjust bolus parameters, and validate design outputs, leading to inefficiencies and delays. Furthermore, the need for clinicians to navigate multiple software platforms increases the risk of errors, while lack of integration with clinical workflow systems and radiation therapy planning software results in bottlenecks. Additionally, maintaining quality control and regulatory compliance in bolus design, such as ensuring accurate thickness measurements and deviation validation, is often cumbersome. In time-sensitive radiation therapy environments, such inefficiencies can delay treatment, impacting on patient care, safety, regulatory compliance, and the like.

Accordingly, the present disclosure provides systems and methods that integrate automated scripting and workflow optimization to address challenges in the ordering and manufacturing of 3D-printed bolus medical devices. By automating the bolus design and validation process, the system eliminates the need for manual imaging imports, third-party software tools, and redundant user input, streamlining workflows, reducing errors, and the like. Integration with radiotherapy treatment planning systems and clinical imaging databases enables seamless retrieval and processing of medical imaging data, eliminating bottlenecks and ensuring timely access to critical information. Advantageously, the systems and methods can incorporate script-driven validation processes, deviation analysis, and automated quality control, ensuring compliance with treatment planning standards and 3D printing accuracy requirements throughout the process. Additionally, automation dynamically guides clinicians and technicians through the bolus ordering process, reducing the risk of errors, and improving efficiency, particularly in time-sensitive radiation therapy workflows. The systems and methods of the present disclosure provide a comprehensive solution that enhances the accuracy, speed, and compliance of 3D-printed bolus ordering and manufacturing, ensuring that patient-specific boluses are delivered quickly and precisely for optimized radiation treatment.

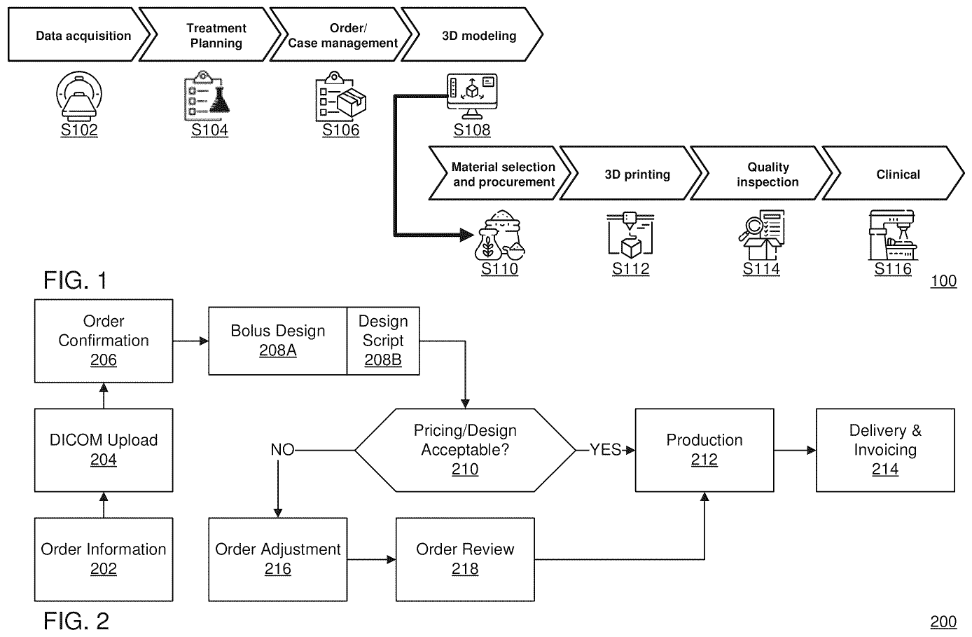

Referring now to the drawings, wherein like reference numerals designate identical or corresponding parts throughout the several views, and more particularly to FIG. 1 thereof, there is shown an ordering and manufacturing workflow 100 for 3D-printed bolus medical devices, according to the present disclosure. In FIG. 1, the ordering and manufacturing workflow 100 illustrates a structured and integrated process designed to streamline the creation of patient-specific boluses for radiation therapy. The workflow begins at S102 for data acquisition, where patient imaging data, typically from computed tomography (CT) scans, magnetic resonance imaging (MRI) scans, and the like, are generated, obtained, and the like, and prepared for further processing. At S104, treatment planning is performed, during which clinicians define radiation parameters, bolus parameters, and the like, to ensure optimal dose distribution, and the like. Order and case management occur at S106, where order details are recorded, patient data is linked to the bolus design process, and automated checks are performed to advantageously ensure bolus design consistency, and the like.

At S108, 3D modeling is conducted to generate a patient-specific bolus design based on anatomical parameters, radiation therapy parameters, and the like, which can advantageously utilize automated tools to optimize the bolus shape for improved fit, dose conformity, and the like. Once the bolus design is finalized, the workflow proceeds to step S110, where material selection, procurement, and the like, occur, advantageously, ensuring appropriate 3D printing materials, and the like, are selected based on prescribed treatment parameters, regulatory standards, biocompatibility considerations, and the like.

In step S112, the bolus is 3D printed using an optimized additive manufacturing process tailored for medical applications, and the like. Advanced print settings, such as layer thickness, infill density, print orientation, and the like, are advantageously adjusted to achieve suitable mechanical properties, and the like. Following printing, step S114 involves quality inspection, where the bolus undergoes rigorous evaluation using scanning, dimensional analysis, material integrity checks, and the like, to advantageously verify compliance with clinical specifications, and the like. The next stage, step S116, involves the clinical application of the bolus, where the validated bolus is delivered to the treatment facility, fitted to the patient, and used during radiation therapy to enhance dose accuracy, treatment efficacy, and the like.

The workflow 100 depicted in FIG. 1 advantageously provides a fully integrated and automated solution that minimizes manual interventions, reduces errors, and ensures efficient, repeatable, and high-quality production of 3D-printed bolus medical devices, and the like. Such streamlined approach advantageously enhances traceability, regulatory compliance, patient safety, and the like, while expediting the overall treatment workflow.

FIG. 2 shows an ordering and manufacturing decision workflow system 200 for 3D-printed bolus medical devices, according to the present disclosure. In FIG. 2, the ordering and manufacturing decision workflow system 200 illustrates a structured, automated, and iterative process for managing the intake, design, validation, production, and delivery of a bolus medical device, and the like. The workflow begins at order information 202, where patient treatment details, and the like, are entered into the system, linking the order to specific clinical requirements while ensuring compatibility with radiotherapy planning software, and the like.

At Digital Imaging and Communications in Medicine (DICOM) upload 204, files including patient imaging data, such as computed tomography (CT) scans, magnetic resonance imaging (MRI) scans, and the like, and radiation therapy parameters, and the like, are uploaded and integrated into the workflow, and the like. Such imaging files advantageously provide the anatomical foundation and requirements for patient-specific bolus customization, and the like. Order confirmation 206 follows, performing an initial validation to ensure that all required data, including imaging parameters, treatment parameters, and the like, have been provided before initiating the design phase, and the like.

At bolus design 208A, a patient-specific bolus model is computationally generated based on anatomical parameters, radiation therapy parameters, and the like, utilizing an automated workflow to optimize bolus geometry for proper fit, dose conformity, material properties, and the like. The process incorporates design script 208B, which executes automated design functions, reducing manual interventions while ensuring precise bolus customization, and the like. The design script 208B advantageously applies smoothing algorithms, Boolean subtraction techniques for anatomical conformity, automated labeling features, and the like, to enhance production efficiency, and the like.

Once the bolus design is generated, pricing/design evaluation 210 is conducted to determine whether the model meets clinical criteria, regulatory criteria, financial criteria, and the like. If the pricing or design meets the employed standards, the workflow proceeds to production 212. If the pricing or design does not meet the employed standards, the workflow proceeds to order adjustment 216, where modifications are made based on clinician feedback, physicist feedback, administrative feedback, and the like. Order review 218 follows, where a final verification advantageously ensures that the adjusted design meets all predefined requirements, including radiation attenuation properties, blocking/non-blocking functionalities, and the like, before moving forward to production, and the like.

If the bolus passes order review 218, the workflow advances to production 212, where the bolus is manufactured using an optimized 3D printing process, and the like. Advanced print parameters, including layer thickness, infill density, multi-material construction, material selection, and the like, are advantageously applied to ensure structural integrity, biocompatibility, clinical efficacy, and the like. Following fabrication, the workflow transitions to delivery and invoicing 214, where the final product is packaged, shipped to the clinical site, and the corresponding invoicing documentation is processed, and the like.

The ordering and manufacturing decision workflow system 200 of FIG. 2 advantageously ensures a seamless and automated process with validation before production, minimizes errors, reduces inefficiencies, enhances traceability, and the like. By integrating computational bolus design automation, real-time validation, structured order review, and the like, the system advantageously supports an end-to-end, zero-touch workflow that enhances accuracy, regulatory compliance, treatment efficacy, and the like, while enabling scalable manufacturing for centralized or Point-of-Care (POC) production, and the like.

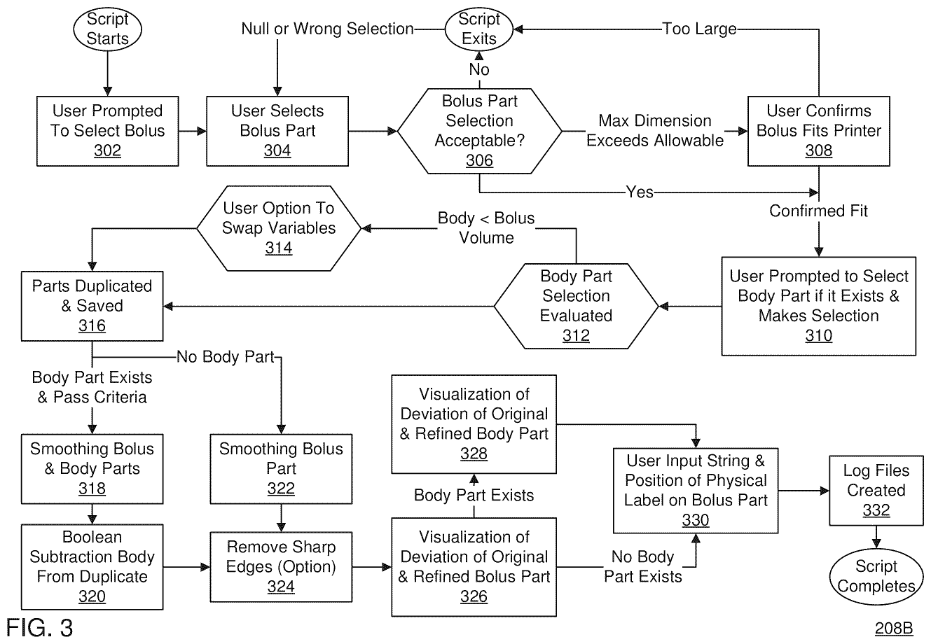

FIG. 3 shows a bolus design script workflow 208B for generating a patient-specific bolus in an automated and iterative manner, according to the present disclosure. In FIG. 3, the bolus design script workflow 208B illustrates a structured computational process executed within a suitable application (e.g., Materialise 3-Matic Medical application, etc.), enabling precision-driven 3D modeling, error detection, quality control, and the like. The bolus design script workflow 208B advantageously enhances repeatability, accuracy, and efficiency by incorporating automated part validation, mesh refinement, Boolean operations, and deviation mapping, and the like.

The bolus design script workflow 208B begins when the script starts, prompting the user to select a bolus part at 302, and the like. At 304, the user selects the bolus part, triggering 306, where an automated evaluation determines whether the selected bolus part is acceptable based on dimensional constraints, volume criteria, mesh integrity, and the like. If the bolus part exceeds the maximum allowable dimension, the user is notified at 308 and prompted to confirm whether the bolus fits within the validated 3D printer build volume, and the like. If the bolus does not fit, the script exits to prevent the use of non-compliant parts, and the like. If the selection is null or incorrect, the script returns to 304. If the bolus meets dimensional constraints, the process advances to 310, where the user is prompted to select a body part if applicable, and the like. At 312, the selected body part undergoes evaluation for volume differentials, overlap detection and compatibility with the bolus, and the like. If the body part volume is smaller than the bolus volume, the user is given an option to swap variable assignments at 314, ensuring proper part designation, and the like. Otherwise, execution will transfer to 316.

At 316, the bolus and body parts are duplicated and stored in an “Originals” folder, retaining unmodified copies for traceability, quality assurance, and regulatory compliance, and the like. If a body part has been selected and meets selection criteria, the script executes 318, performing smoothing operations on the bolus and body parts to refine mesh quality and remove artifacts, and the like. At 320, a Boolean subtraction operation is performed to remove the body part from the bolus duplicate, ensuring proper anatomical conformity, and the like, and execution moves to 324.

If no body part has been selected and meets selection criteria, the script proceeds directly to smoothing the bolus part at 322, where segmentation artifacts are refined to enhance print quality and ensure clinical accuracy, and the like, and execution moves to 324. At 324, the user is given an option to remove sharp edges, further optimizing patient comfort, bolus fit, and the like.

The script then proceeds to 326, where a visualization of deviation between the original and refined bolus part is generated. Such visualization employs color mapping and deviation calculations to ensure surface conformity within a predetermined tolerance, and the like (e.g., around ±1 mm, etc.). If no body part has been selected and meets selection criteria, the script proceeds to 330. If a body part has been selected and meets selection criteria, the script proceeds to 328, where a visualization of deviation between the original and refined body part is generated to ensure high-quality adaptation and minimal deviation from clinical requirements, and the like. The script then proceeds to 330.

At 330 the user is prompted to input a string identifier for a physical label and specify the placement position on the bolus part, ensuring traceability, compliance with labeling standards, manufacturing consistency, and the like. At 332, the script generates log files capturing employed metadata, including design parameters, surface deviation analysis, processing time, user interactions, and the like. Once the logs are recorded, the script is completed.

The bolus design script workflow 208B of FIG. 3 advantageously ensures a highly structured and automated process for 3D bolus design, leveraging real-time quality control, parameter-based mesh refinement, automated Boolean operations, deviation analysis, metadata logging, and the like. By integrating computational validation, scripting automation, and error mitigation functions, the workflow minimizes manual intervention, optimizes treatment planning efficiency, and enhances patient-specific customization, and the like.

The bolus design script workflow 208B of FIG. 3 can operate within a software architecture system where a user interacts with a 3D modeling application (e.g., 3-Matic Application, etc.) executing the bolus design script workflow 208B (e.g., as a Python script, etc.) for automated bolus generation. The script 208b runs on any suitable operating system (e.g., Windows OS, etc.) utilizing system application programming interfaces (APIs) and services to perform 3D modeling, validation, processing, and the like. The system can also connect to external resources and/or services for data storage or integration, and the like. Such structured flow ensures efficient user input, automated processing, and streamlined bolus customization.

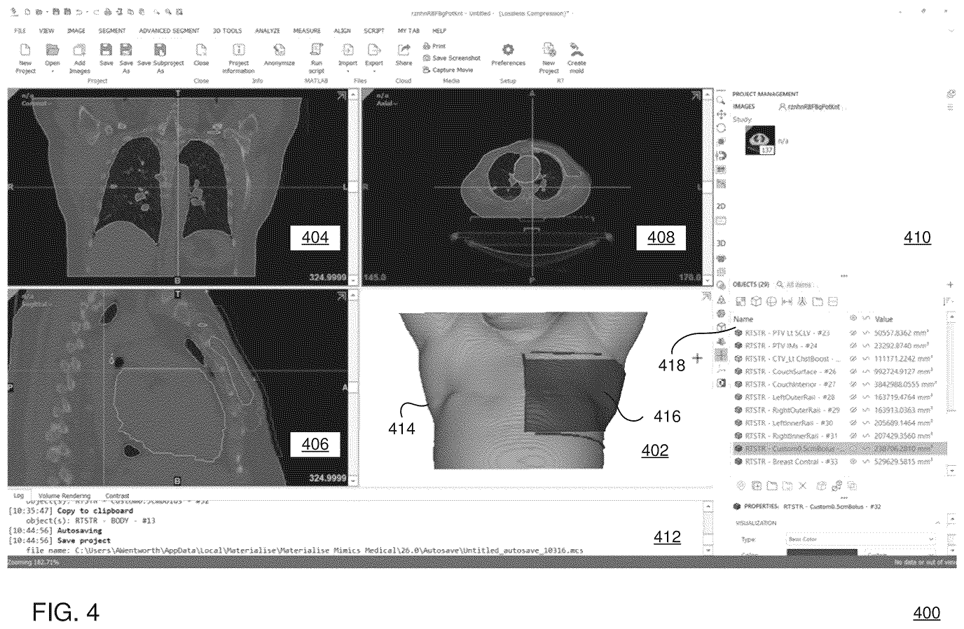

FIGS. 4-14 show a bolus design script graphical user interface (GUI) 400 for the bolus design script workflow of FIG. 3, according to the present disclosure. In FIG. 4, the bolus design script graphical user interface (GUI) 400 is shown, illustrating the structured, automated, and iterative workflow for patient-specific bolus generation, according to the present disclosure. The interface integrates multiple visualization panels, project management tools, object selection options, and logging functions to streamline the bolus creation process. A display area 402 provides a 3D rendering of the patient's anatomical structure, including a body 414 and corresponding bolus 416. The bolus 416 is represented as a distinct overlay on the patient's body 414, indicating the customized bolus placement for radiation therapy. The display area 402 supports interactive manipulation, ensuring precise bolus fit and conformance to anatomical contours. A coronal view 404, sagittal view 406, and axial view 408 present cross-sectional imaging from the patient's CT scan, enabling users to analyze anatomical structures and confirm bolus placement relative to critical treatment regions. These views advantageously facilitate a comprehensive assessment of the patient's anatomy to optimize the bolus design. A project management panel 410 allows users to manage imaging datasets, including Digital Imaging and Communications in Medicine (DICOM) files, radiation therapy structure (RTSTRUCT) files, and segmentation data. Panel 410 organizes imaging data into structured lists, enabling efficient access to boluses and body structures while maintaining traceability. A log panel 412 continuously records system events, user actions, and processing steps, ensuring traceability for quality assurance and regulatory compliance. Logged data includes timestamps, file paths, user selections, and script execution details, which advantageously support debugging and validation.

The interactive object selection tool 418 enables users to manipulate, refine, and validate the bolus structure through interactive selection and processing options. The tool 418 allows users to select, duplicate, or modify structures, ensuring the generated bolus meets clinical and manufacturing specifications. By integrating multi-view imaging analysis, automated structure selection, 3D modeling, and real-time logging, GUI 400 enhances precision, repeatability, and efficiency in bolus customization. The bolus design script workflow ensures that each step, from data input to finalization, is executed with high accuracy and minimal manual intervention, supporting seamless integration into radiation therapy planning.

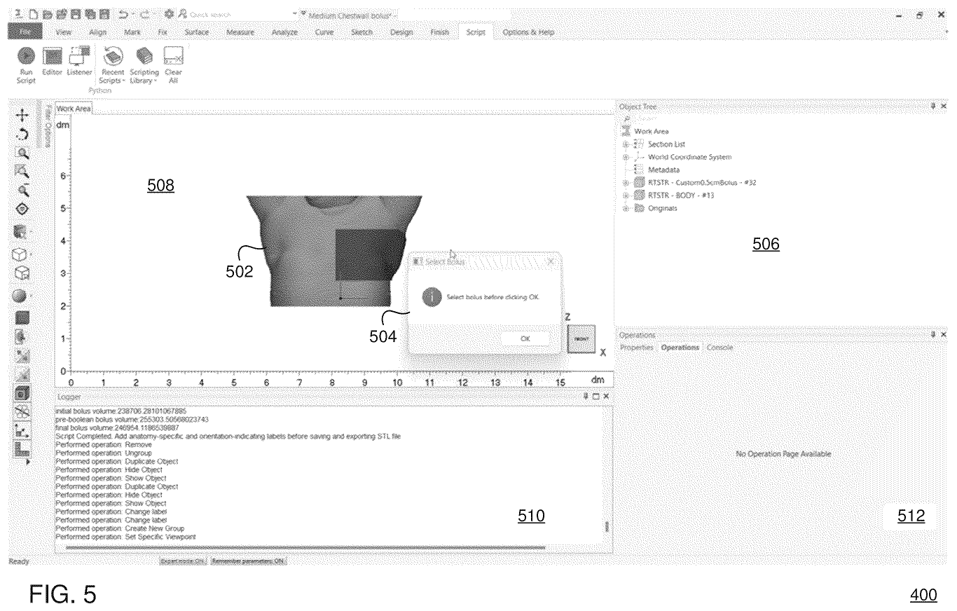

In FIG. 5, the GUI 400 advantageously integrates 3D visualization, interactive selection prompts, and structured data management tools to ensure precision-driven bolus design. A 3D display area 508 presents a rendered view of the patient's anatomical structure, including a body and corresponding bolus 502 overlaid on the target treatment region. Such visualization enables users to confirm bolus placement and alignment relative to anatomical features. The display area 508 supports interactive manipulation to optimize bolus conformance and treatment planning. A selection prompt 504 appears as a pop-up window, instructing the user to “Select bolus before clicking OK” to proceed with the next processing step. Such interactive validation ensures that the correct structure is chosen before executing subsequent smoothing, Boolean subtraction, and labeling operations. The selection prompt 504 mitigates errors by preventing unintended script execution without a properly designated bolus. An object tree panel 506 organizes project data, listing all imported and generated structures, including the body model and bolus file, along with metadata. Such structured hierarchy allows users to efficiently access, modify, and validate the selected objects before final processing. A log panel 510 continuously records system actions, user inputs, and automated script operations, ensuring traceability for quality assurance, debugging, and regulatory compliance. Logged data includes the initial bolus volume, pre-bolus adjustments, final bolus volume, performed operations, and unique labeling instructions. An operations panel 512 provides additional functionality for modifying and executing commands within the software environment. Section 512 remains inactive until a valid bolus selection is confirmed, at which point relevant operations are displayed.

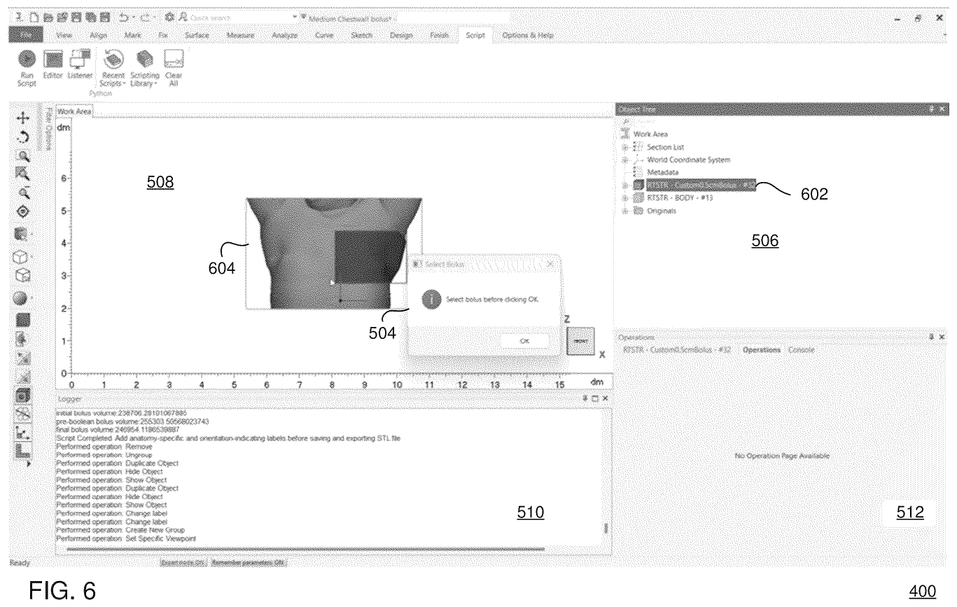

In FIG. 6, the GUI 400 shows the 3D display area 508 presenting a rendered patient model with an overlaid selected bolus 604, allowing users to confirm bolus selection. A highlighted entry 602 represents the selected bolus structure that represents the anatomical model used for reference and Boolean operations. Such hierarchical organization enables efficient object selection, modification, and validation before processing. The log panel 510 records system operations, user actions, and script execution details. Logged data includes initial bolus volume, pre-bolus modifications, final bolus volume, and performed operations, ensuring traceability, quality control, and regulatory compliance. Operations panel 512 provides operational information when available.

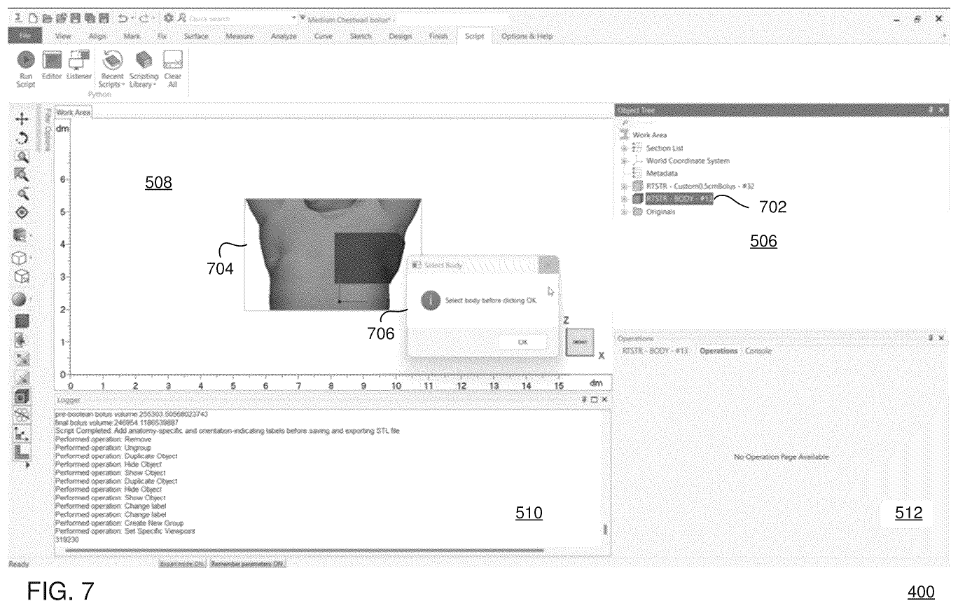

In FIG. 7, the GUI 400 shows the 3D display area 508 presenting a selected body with an overlaid bolus 704, allowing users to confirm body selection. A selection prompt 706 appears, instructing the user to “Select body before clicking OK,” ensuring that the correct anatomical reference structure body is designated before proceeding. The object tree panel 506 includes a highlighted body structure 702, representing the anatomical model used for reference and Boolean operations. Such hierarchical organization enables efficient object selection, modification, and validation before processing. The log panel 510 records system operations, user actions, and script execution details, including pre-bolus volume, final bolus volume, and performed operations. An operations panel 512 provides additional functionality when applicable.

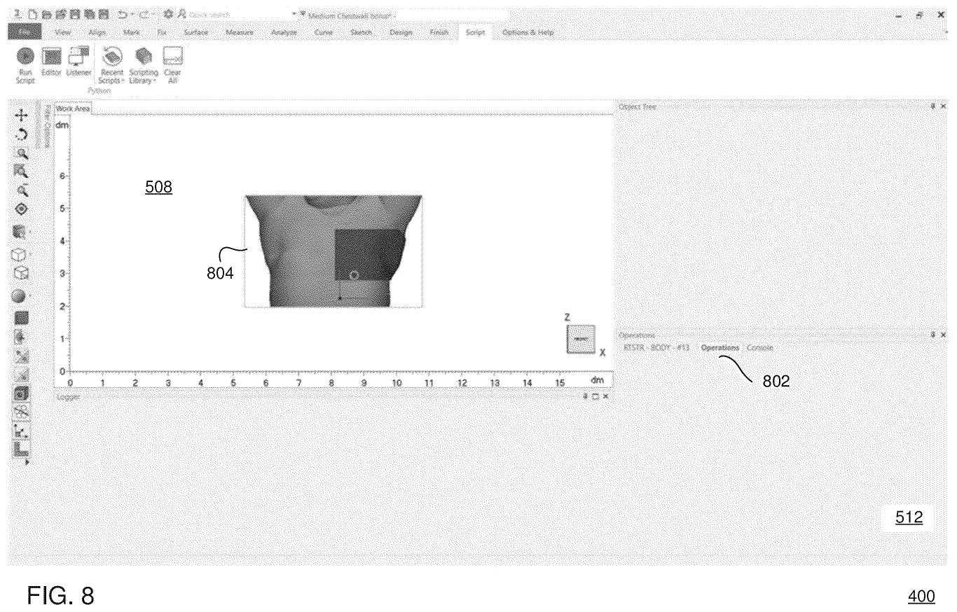

In FIG. 8, the GUI 400 shows the 3D display area 508 rendering the selected patient body model with an overlaid bolus 804, providing visual confirmation. Operations panel 512 is running operations 802 on the selected body structure and bolus.

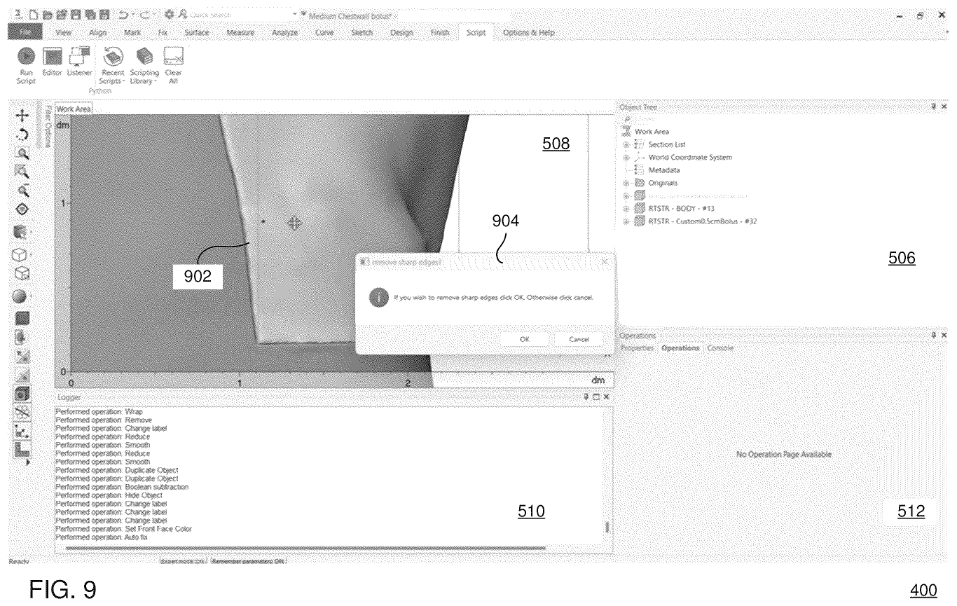

In FIG. 9, the GUI 400 shows the 3D display area 508 with a zoomed-in view of the bolus structure 902, highlighting the finer details of its contours and edges. The object tree panel 506 remains visible, maintaining a structured list of imported and generated models for easy reference and modification. A selection prompt 904 appears as a pop-up window, offering the user an option to remove sharp edges, ensuring improved patient comfort and optimized bolus fit. The log panel 510 records performed operations, including wrapping, smoothing, Boolean subtraction, and labeling modifications, providing a detailed history for quality assurance and reproducibility. The operations panel 512 remains accessible for further processing as needed.

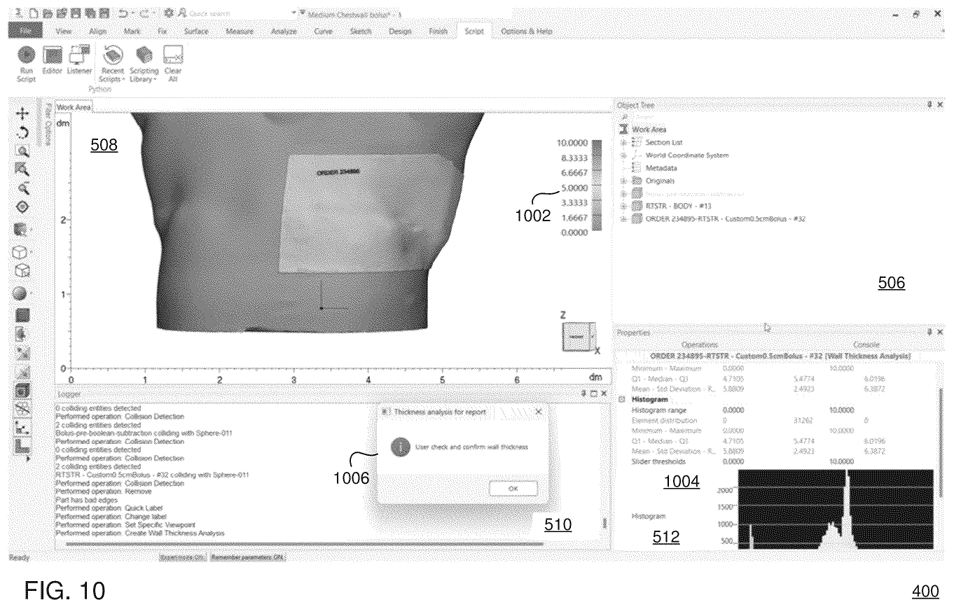

In FIG. 10, the GUI 400 presents the 3D display area 508 with the bolus structure 1002, now labeled with an order number for traceability. The object tree panel 506 maintains an organized hierarchy of project files, including the body model and custom bolus structure. A thickness analysis histogram 1004 appears in the properties panel, providing statistical data on the bolus wall thickness distribution, including minimum, maximum, mean, and standard deviation values. The log panel 510 continues to document performed operations, such as collision detection, labeling, and thickness analysis execution, ensuring traceability and validation. A user prompt 1006 appears, instructing the user to confirm the wall thickness analysis results before proceeding with further processing or finalization. The operations panel 512 displays additional analytical data related to the thickness assessment.

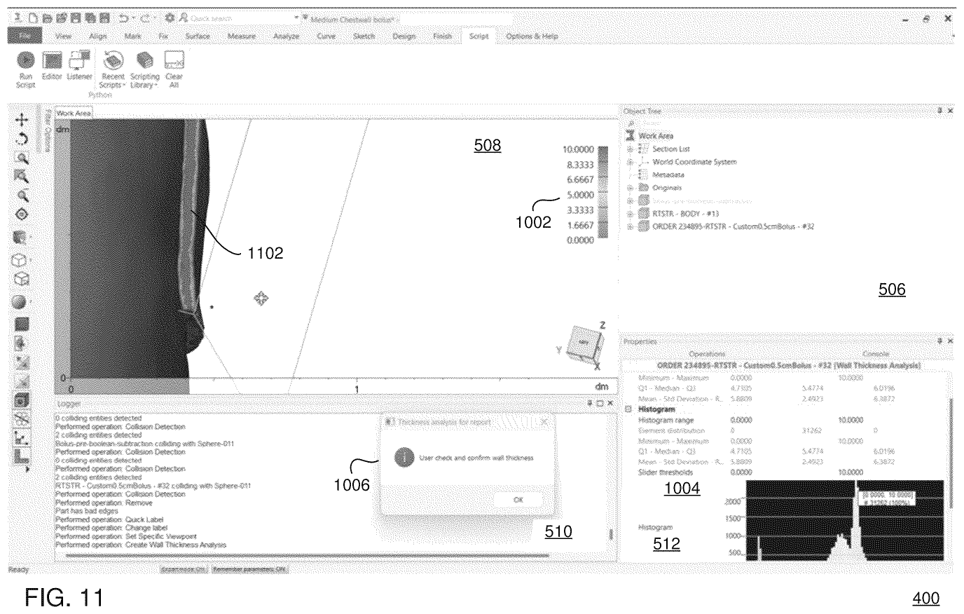

In FIG. 11, GUI 400 presents a detailed thickness analysis of the bolus structure, ensuring accuracy in patient-specific customization. The 3D display area 508 provides a side view of the bolus, enabling visualization of the thickness distribution along the patient's anatomy. The color-coded scale 1002 represents thickness variations, assisting in the identification of inconsistencies or deviations. The histogram 1004 quantifies the thickness distribution, displaying minimum, maximum, and average values for verification. Such statistical analysis allows users to validate bolus conformity to treatment specifications. The thickness analysis confirmation prompt 1006 prompts user verification before proceeding, ensuring manual oversight in critical validation steps. The object tree panel 506 organizes hierarchical project data, listing the bolus and body models for easy access and modification. The log panel 510 records real-time processing details, including thickness evaluation steps and system actions for traceability. The operations panel 512 provides additional analysis tools and settings related to thickness measurement. The side view of bolus 1102 in the display area 508 highlights the thickness consistency along the treatment site, ensuring even coverage for radiation therapy. By integrating automated thickness analysis, real-time visualization, and user confirmation steps, the GUI 400 enhances precision, quality control, and clinical validation in bolus design.

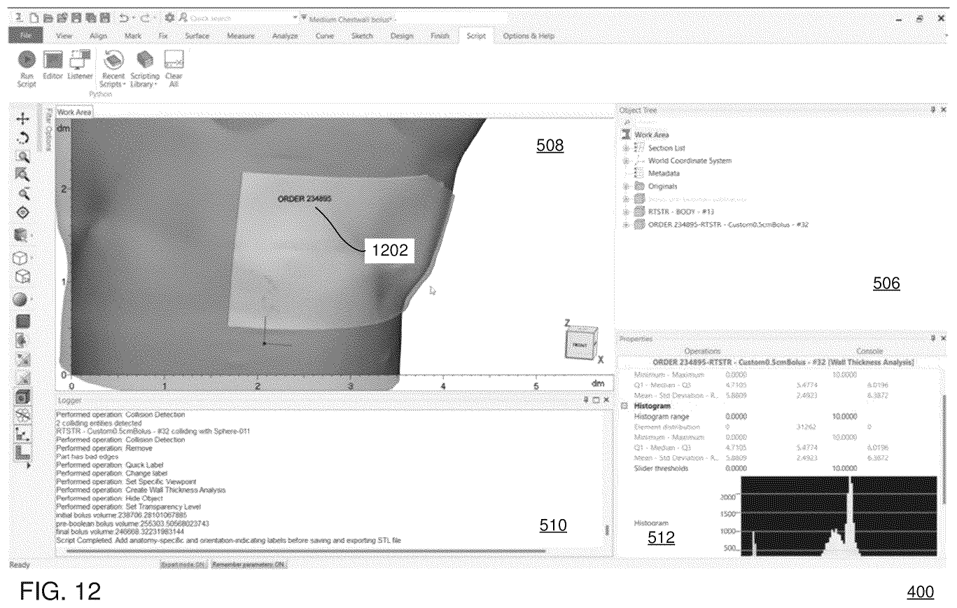

In FIG. 12, the GUI 400 introduces an applied label 1202 on the bolus, ensuring traceability for manufacturing and clinical verification. The 3D display area 508 now highlights the finalized bolus design in a transparent mode with its assigned order number, confirming its integration into the workflow. The histogram 512 continues to provide a detailed thickness analysis, validating uniformity. The log panel 510 records the finalization steps, including transparency settings and labeling operations, ensuring compliance with quality control standards.

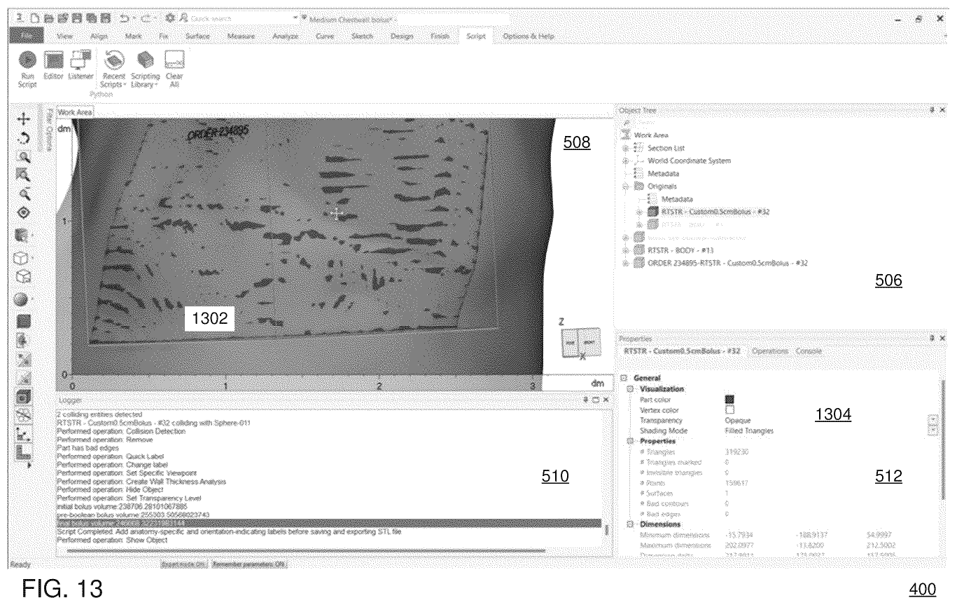

In FIG. 13, the GUI 400 shows the 3D display area 508 introducing a color-coded deviation map 1302, highlighting discrepancies between the refined bolus and the original anatomical surface. Such visualization enables precise assessment of surface variations, ensuring conformance to clinical specifications. The properties panel 1304 provides detailed metadata, including triangle count, shading mode, and transparency settings, facilitating mesh validation. The log panel 510 records additional processing steps, confirming transparency adjustments and deviation analysis before final validation and export.

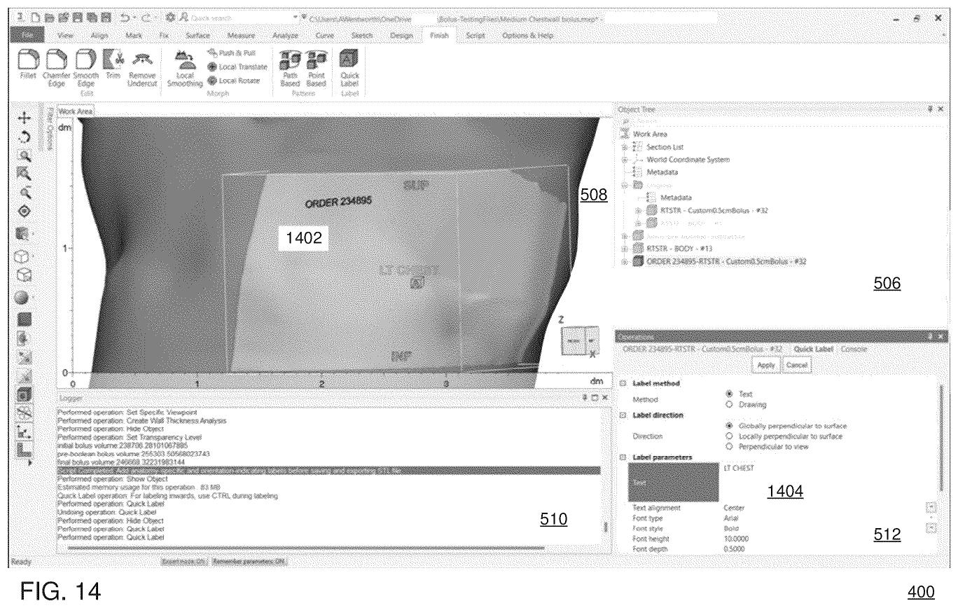

In FIG. 14, the GUI 400 shows a Quick Label feature in the operations section 1404, where label settings, such as text alignment, font type, style, height, and depth are configured. The 3D display area 508 presents the bolus with embedded labels, including “ORDER 234895” and “LT CHEST,” confirming proper label placement. The object tree panel 506 organizes the bolus and body structures, while the log panel 510 records label application operations. The console panel 512 provides system feedback, ensuring successful execution of the labeling operations.

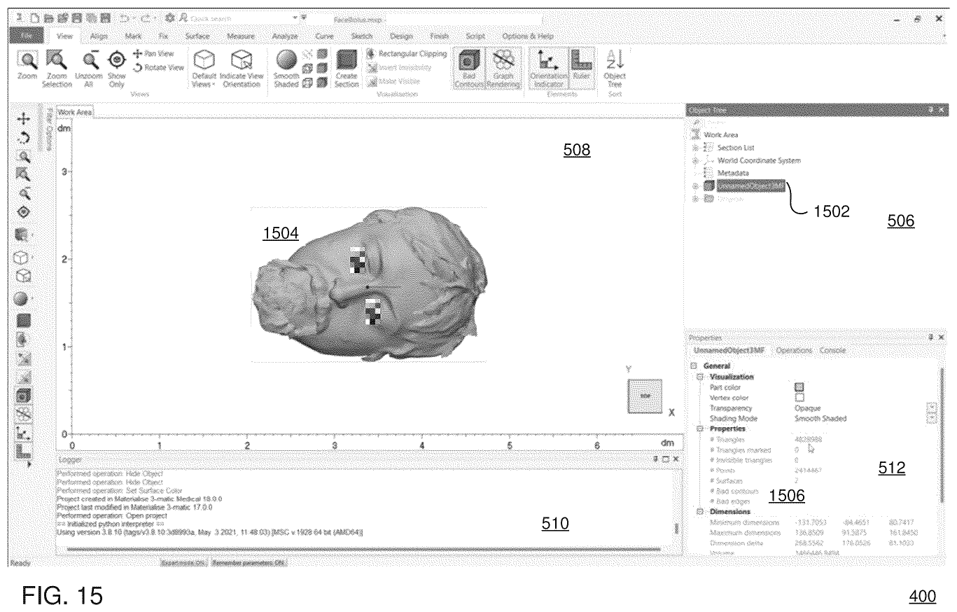

FIGS. 15-22 show a further bolus design script graphical user interface (GUI) for the bolus design script workflow of FIG. 3, according to the present disclosure. In FIG. 15, the GUI 400 shows the 3D display area 508 presenting a patient-specific surface scan 1504, which has been imported into the workflow for bolus customization. The object tree panel 506 lists the imported model 1502, confirming its integration. The properties panel 512 displays mesh attributes 1506, ensuring structural evaluation before further processing. The log panel 510 records the object's importation and processing information for validating workflow execution.

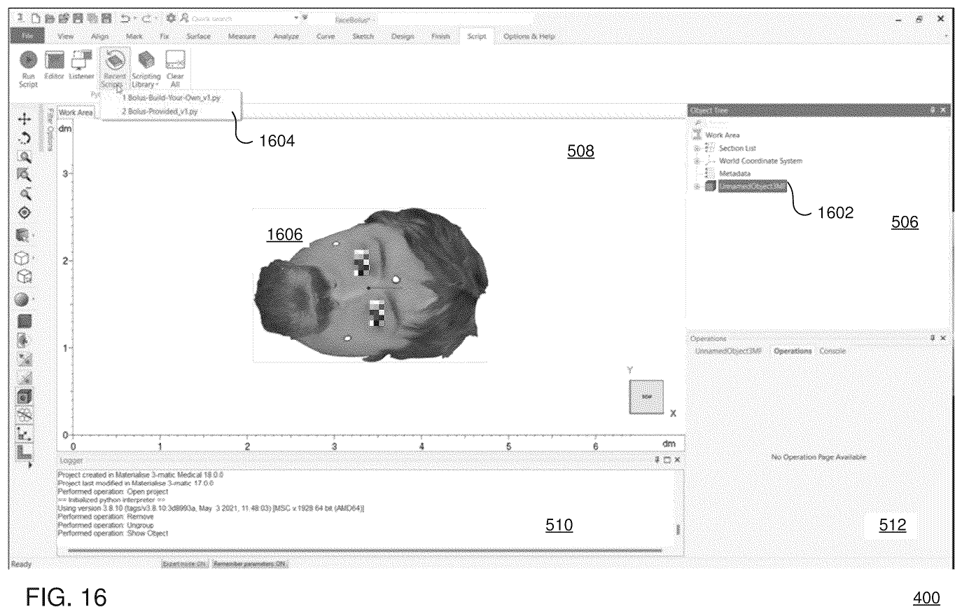

In FIG. 16, the GUI 400 shows the 3D display area 508 presenting a custom surface scan 1606 with a reduced triangle count, improving mesh efficiency for bolus creation. The object tree panel 506 lists the imported model 1602, confirming its continued use in the workflow. The scripting menu 1604 displays recent script selections, including “Bolus-Build-Your-Own_v1.py” and “Bolus-Provided_v1.py”, indicating the execution of automation processes. The log panel 510 records advantageous operations, such as project opening, Python script execution, and mesh modifications, validating preprocessing steps. The operations panel 512 remains available for further operations display.

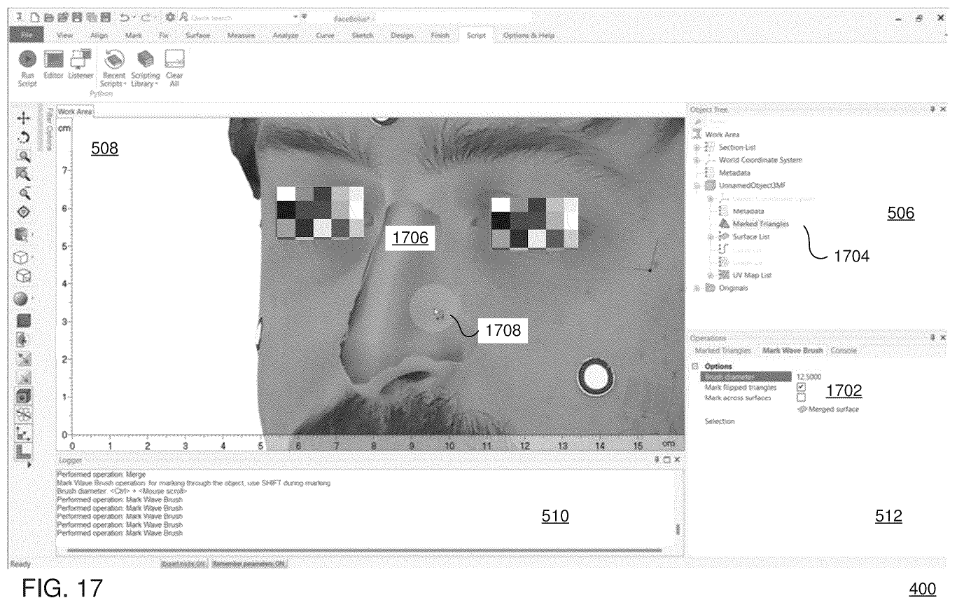

In FIG. 17, the GUI 400 demonstrates the bolus design process with 3D display area 508 showing a close-up of nose region bolus 1706, which is actively being modified using a brush tool 1708. The object tree panel 506 confirms marked triangles 1704 displaying the affected mesh area. The operations panel 512 provides adjustable parameters 1702, including a brush diameter, along with options for marking flipped triangles and marking across surfaces. The log panel 510 documents multiple brush operations, reflecting iterative refinements applied to the bolus 1706.

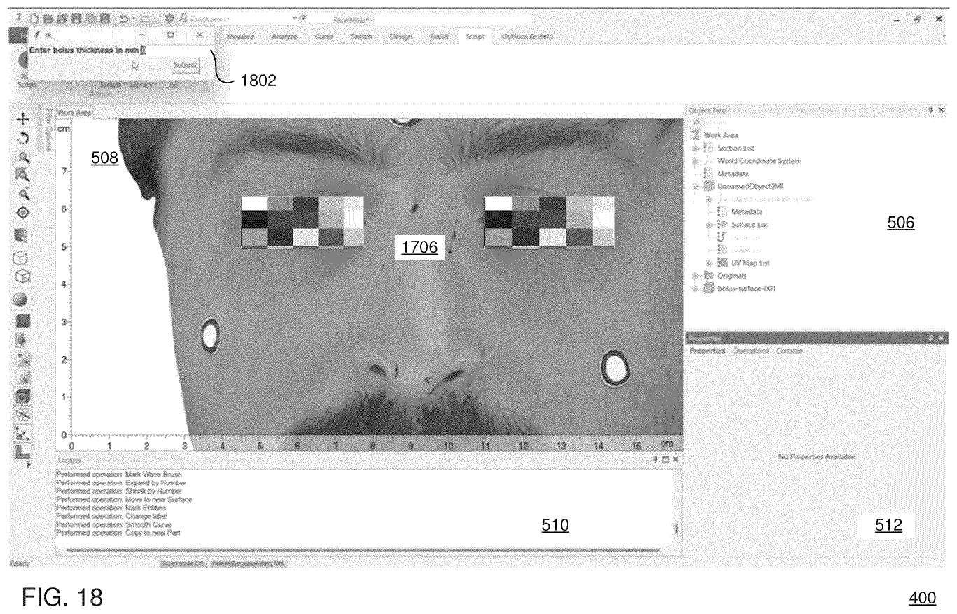

In FIG. 18, the GUI 400 shows the user input step for defining bolus thickness, integrating manual control over the bolus customization process. The bolus thickness input dialog 1802 appears in the interface, prompting the user to enter a thickness value (e.g., in millimeters, etc.). The 3D display area 508 highlights the nose region bolus 1706 with a defined boundary (e.g., a yellow boundary, etc.), indicating the bolus surface selection.

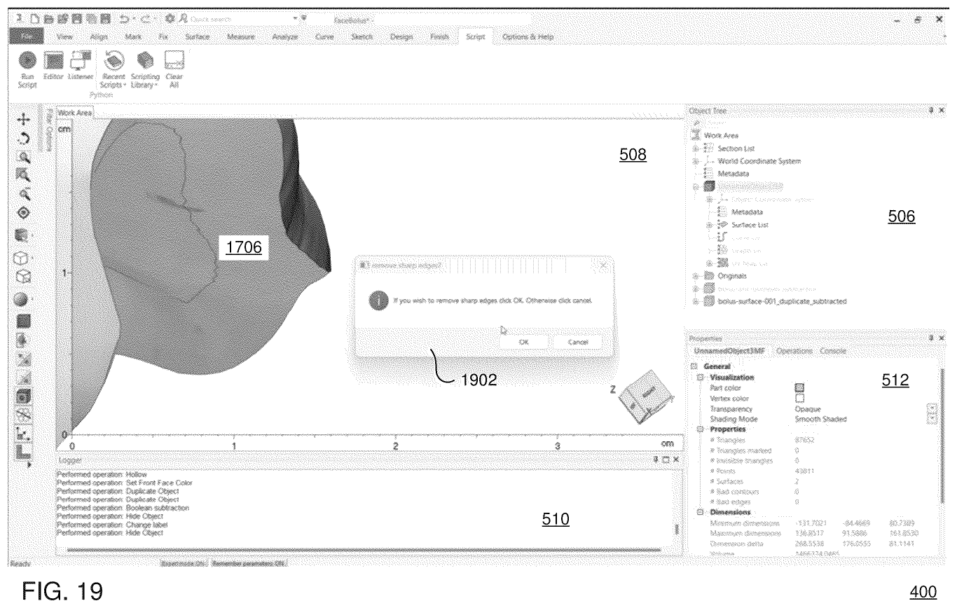

In FIG. 19, the GUI 400 displays a sharp edge removal process, refining the bolus surface for improved fit and patient comfort. A confirmation dialog 1902 appears, prompting the user to approve or cancel the edge smoothing operation. The 3D display area 508 shows the bolus 1706 before the sharp edge removal process.

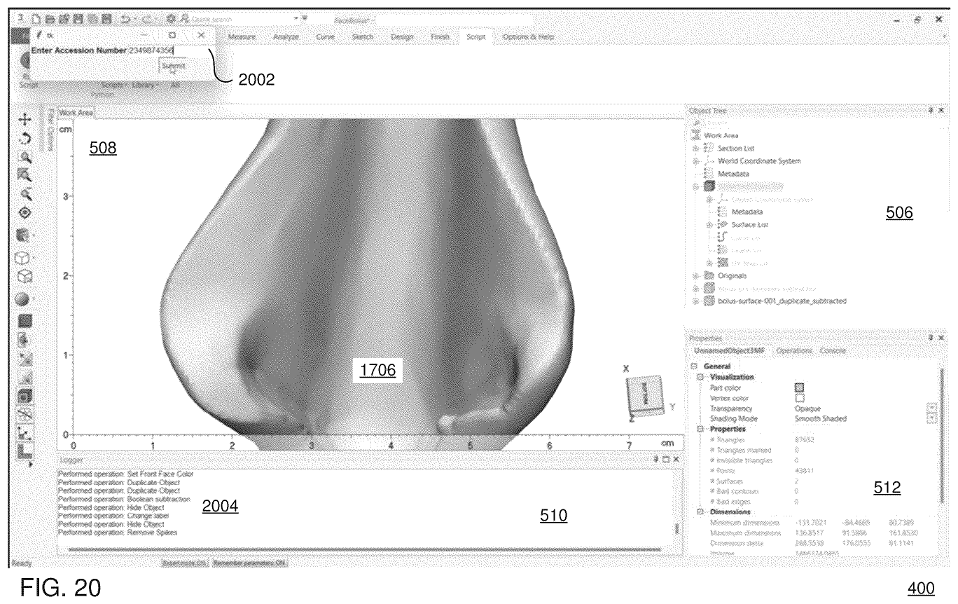

In FIG. 20, the GUI 400 presents the accession number entry step, where a text input field 2002 allows users to input and submit an accession number, and the like, for tracking. The 3D display area 508 displays a smoothened bolus surface over the nasal region, reflecting prior Boolean subtraction and spike removal operations. The object tree panel 506 lists the modified bolus structure, while the log panel 510 and log entry 2004 document performed operations, including front face color setting, object duplication, Boolean subtraction, and spike removal, ensuring traceability in the bolus design workflow.

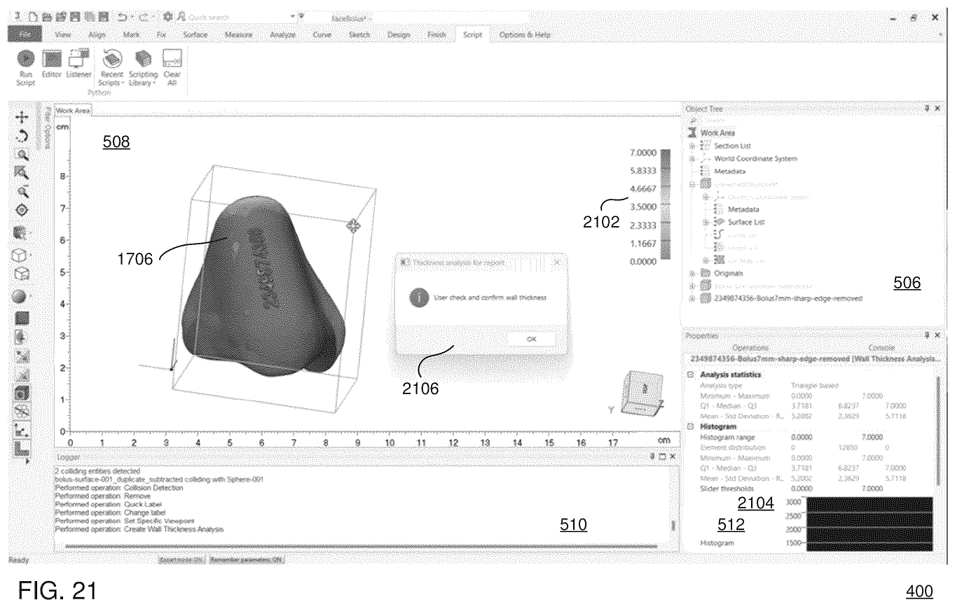

In FIG. 21, the GUI 400 displays the bolus model 1706 with an applied wall thickness analysis, indicated by the color-coded thickness scale 2102 on the right. The accession number remains embossed on the bolus, ensuring traceability. A user prompt 2106 appears, requesting confirmation of the wall thickness analysis results. The properties panel 512 includes thickness analysis statistics 2104, detailing the minimum, maximum, and mean thickness values. The object tree 506 lists the processed bolus file, while the log panel 510 confirms the execution of collision detection, Boolean subtraction, labeling, and thickness analysis operations.

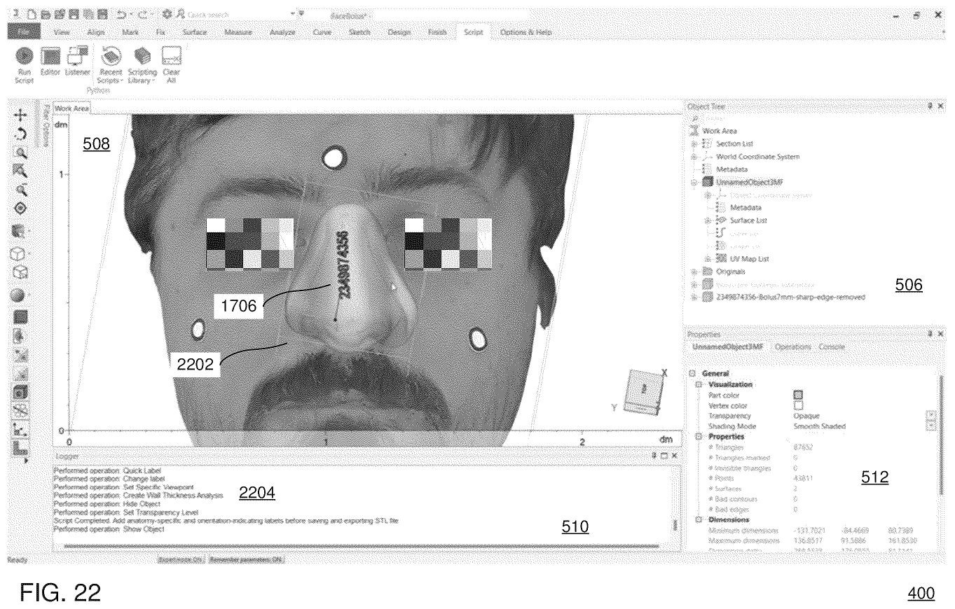

In FIG. 22, the GUI 400 displays the finalized bolus model 1706 fitted to the patient's facial scan, with the Accession Number (e.g., 2349874356, etc.) clearly embossed for identification. A bounding box 2202 confirms the fitting region, ensuring precise alignment. The log panel 510 confirms at 2204 the completion of quick labeling, viewpoint adjustments, transparency settings, and wall thickness analysis. The object tree 506 verifies the integration of the bolus model, while the properties panel 512 provides the finalized mesh details, including number of triangles, ensuring high-resolution accuracy. The bolus model 1706 is now finalized and ready for stereolithography (STL) file export.

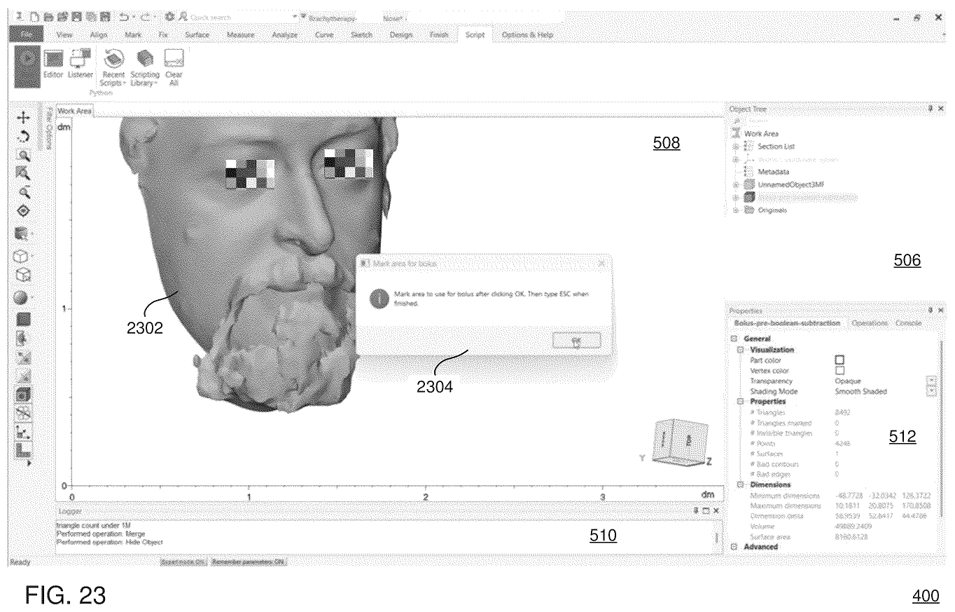

FIGS. 23-40 show a brachytherapy applicator design script graphical user interface (GUI) that can be used with the bolus design script workflow of FIG. 3, according to the present disclosure. In FIG. 23, the GUI 400 displays 3D display area 508, where a patient-specific facial scan 2302 is loaded for brachytherapy applicator design. A system prompt 2304 instructs the user to mark the area for the brachytherapy applicator before proceeding. The log panel 510 records operations such as merging and hiding objects, ensuring procedural traceability. The object tree 506 lists the imported 3D scan, while the properties panel 512 confirms the mesh details, including triangle count and dimensions. This step initiates the bolus customization process, allowing freeform marking to define the treatment area.

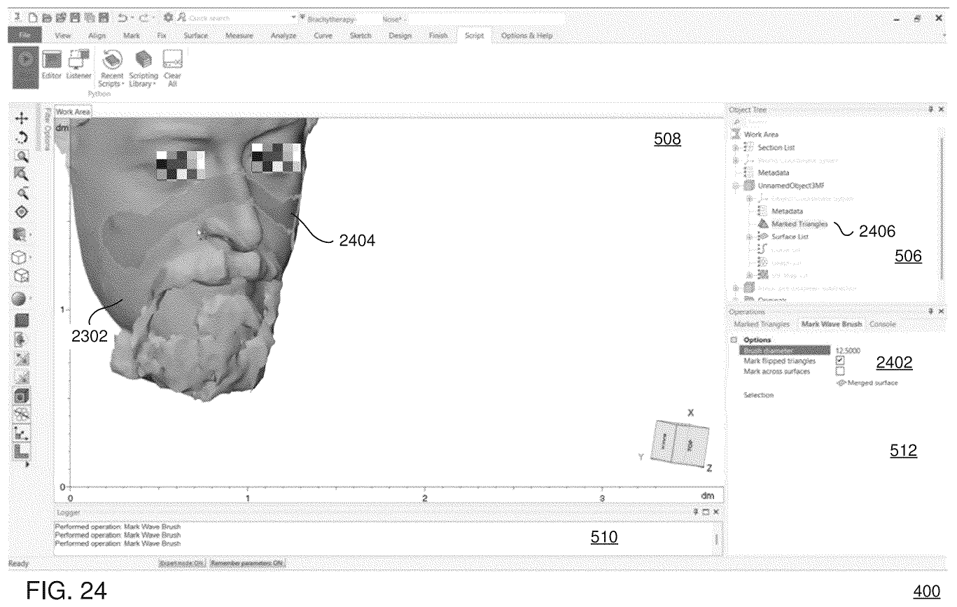

In FIG. 24, the GUI 400 shows the 3D display area 508, where the brachytherapy applicator area 2404 is being marked on the facial scan 2302. The marking tool settings 2402 are visible in the operations panel 512, including brush diameter and options for marking across surfaces. The object tree 506 now contains a marked triangles entry 2406, confirming the active modification region. The log panel 510 records multiple mark wave brush operations, ensuring process traceability. This step refines the bolus boundary, allowing precise customization before generating the final treatment structure.

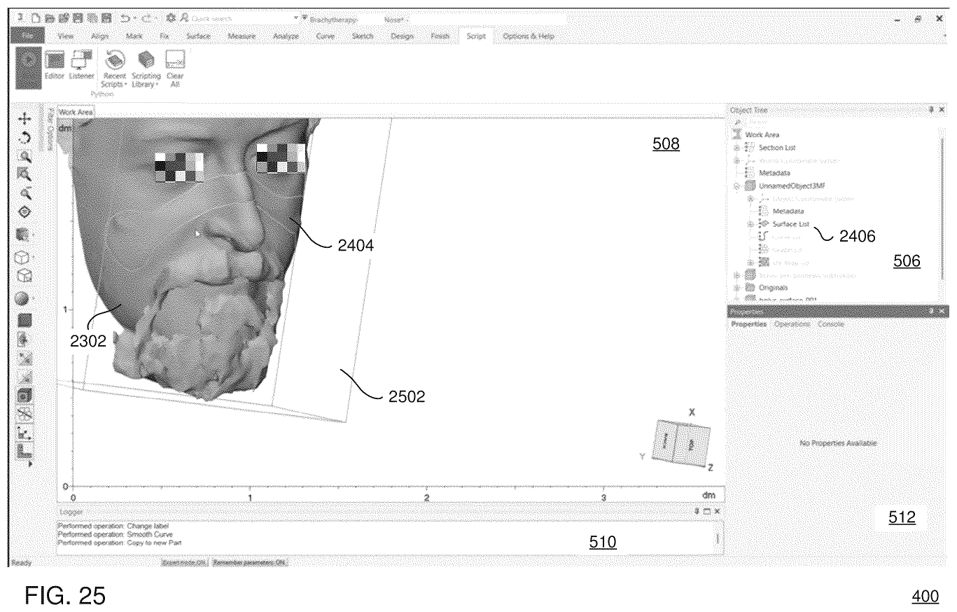

In FIG. 25, the GUI 400 shows the 3D display area 508, where the brachytherapy applicator area 2404 on the facial scan 2302 has undergone a smoothing operation. The bounding box 2502 is visible, indicating the selected region for further refinement. The log panel 510 records actions, such as label change, curve smoothing, and copying to a new part.

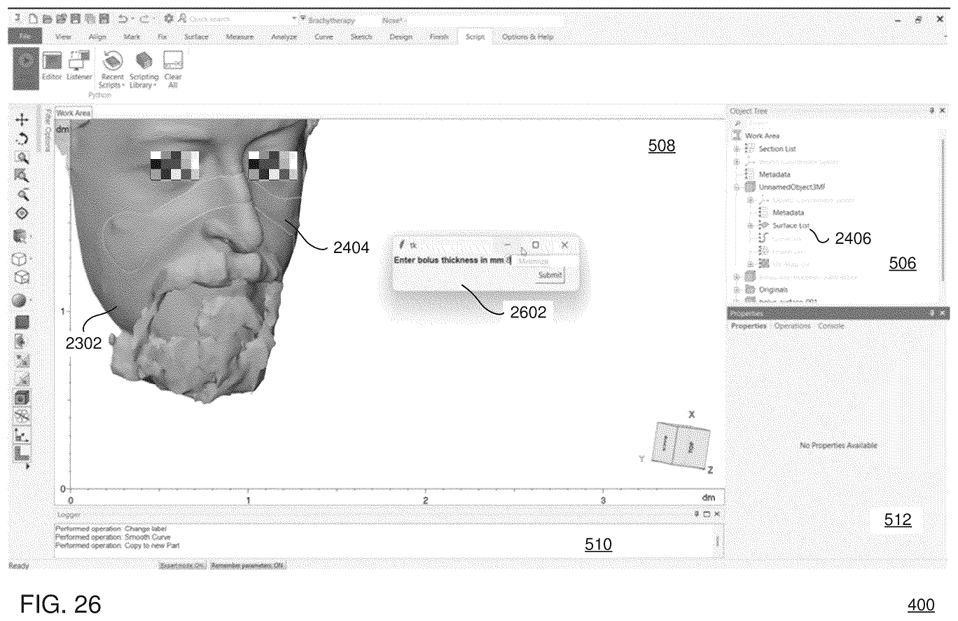

In FIG. 26, the GUI 400 shows an applicator thickness input dialog 2602, allowing the user to specify the desired thickness for the brachytherapy applicator. This interactive step ensures user-defined customization of the applicator thickness before proceeding with further processing.

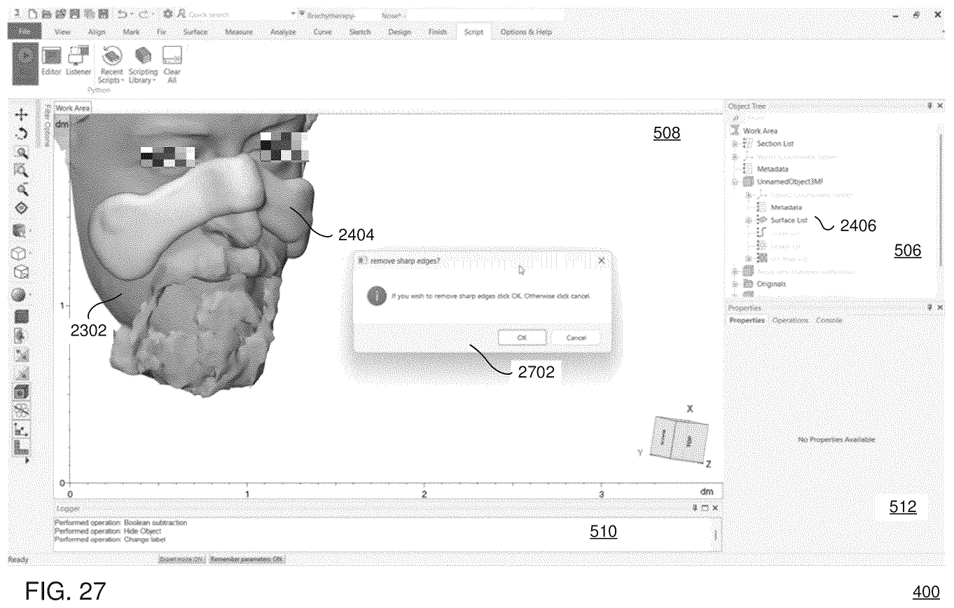

In FIG. 27, the GUI 400 shows a sharp edge removal prompt 2702, allowing the user to confirm whether to smooth out sharp edges on the applicator. The applicator 2404 has now been generated and applied to the scanned face, showing a more defined structure following the thickness input from the previous step.

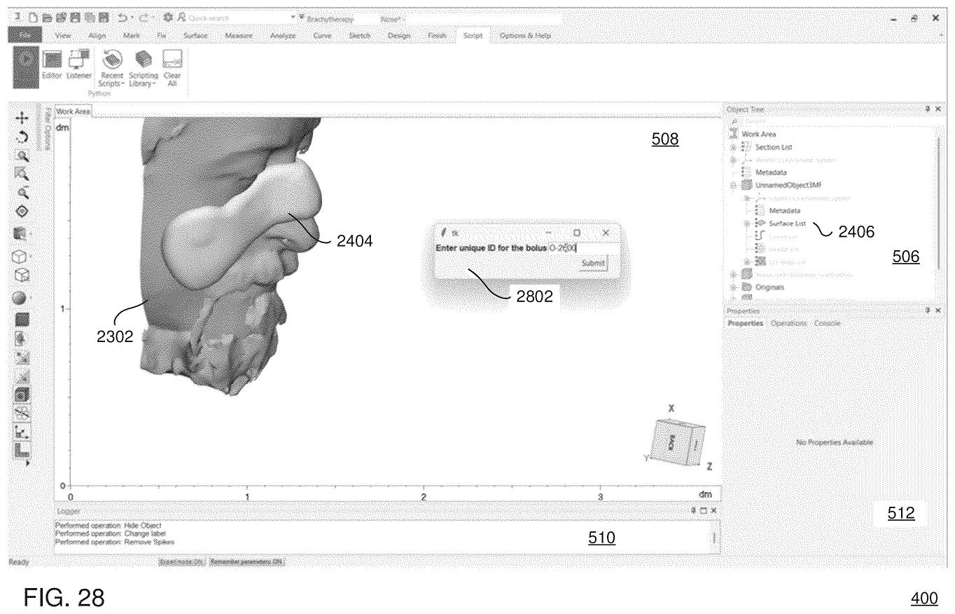

In FIG. 28, the GUI 400 shows a unique ID entry prompt 2802, allowing the user to assign a specific identifier to the brachytherapy applicator 2404. Brachytherapy applicator 2404 is now fully generated and positioned on the scanned facial structure.

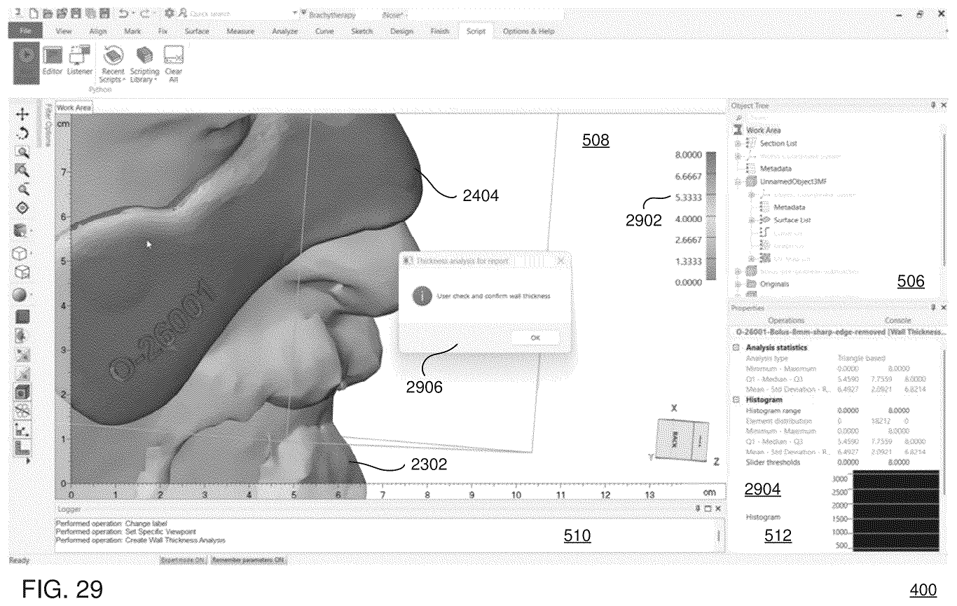

In FIG. 29, the GUI 400 shows thickness analysis 2902 that was performed, displaying a prompt 2906 for user verification. The color-coded scale 2902 represents thickness values across the brachytherapy applicator 2404, ensuring uniformity. A histogram 2904 in the properties panel 512 provides a statistical breakdown of thickness distribution.

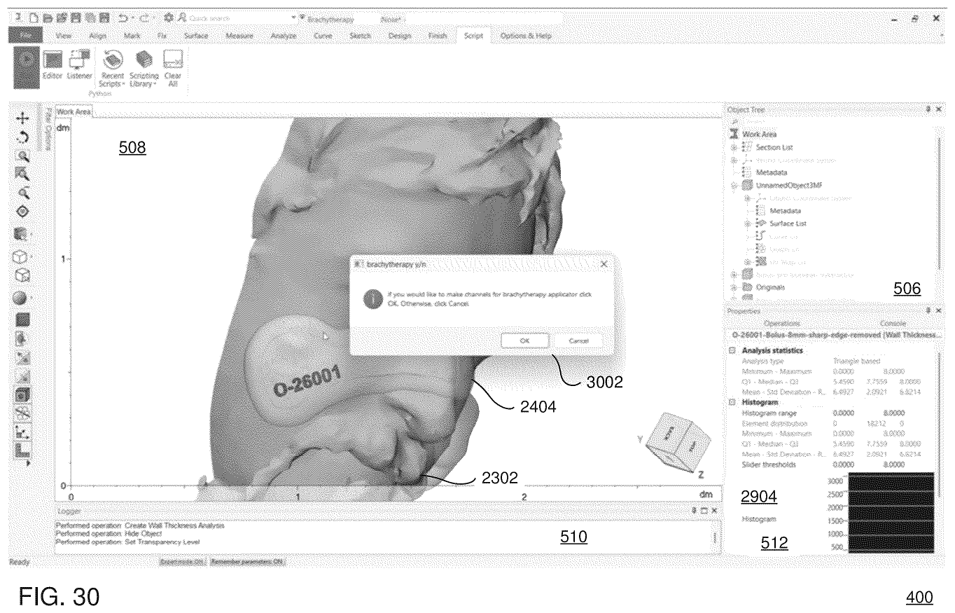

In FIG. 30, the GUI 400 shows a brachytherapy channel creation prompt 3002, allowing the user to confirm or cancel the generation of channels within the brachytherapy applicator 2404 for applicator placement. The thickness analysis 2904 from the previous step remains visible in the properties panel, ensuring dimensional accuracy before proceeding.

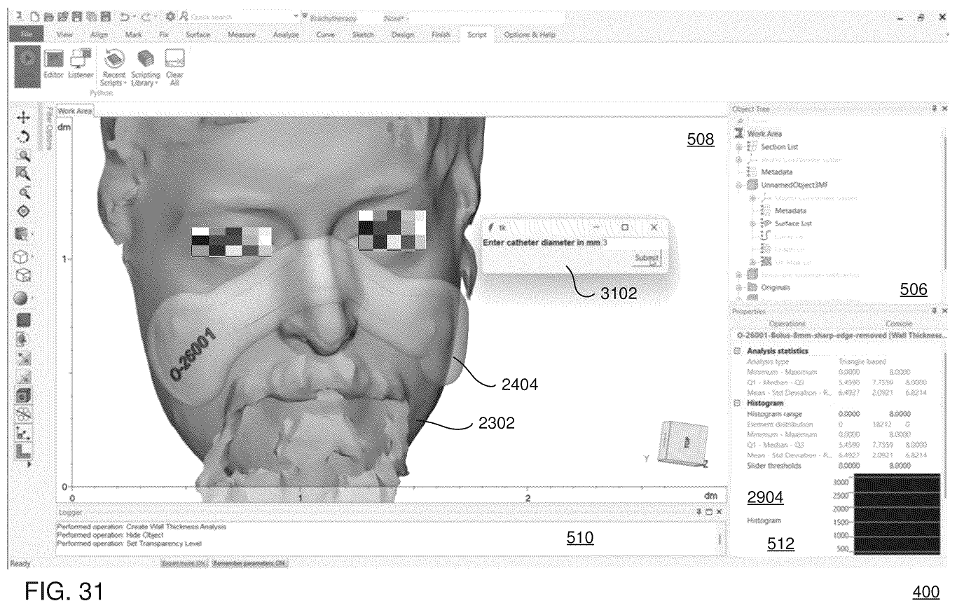

In FIG. 31, the GUI 400 shows an enter catheter diameter input prompt 3102, allowing the user to specify the catheter size (e.g., in millimeters, etc.). This step ensures proper channel dimensions for brachytherapy placement within applicator 2404, which now includes an embossed unique identifier (e.g., O-26001, etc.) for tracking.

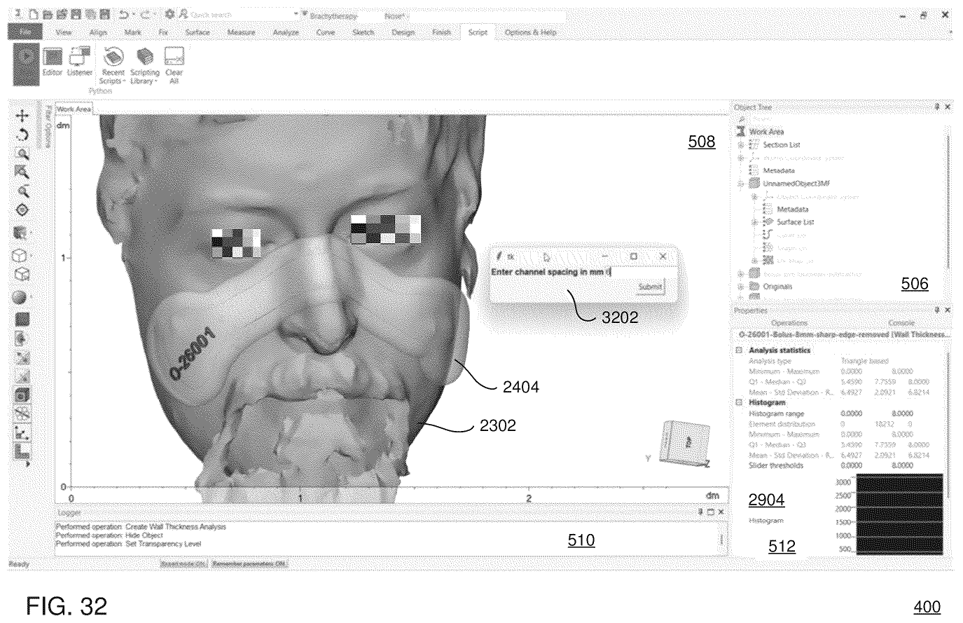

In FIG. 32, the GUI 400 shows an enter channel spacing input prompt 3202, allowing the user to define the distance between brachytherapy channels (e.g., in millimeters, etc.). This ensures precise catheter placement within the applicator 2404.

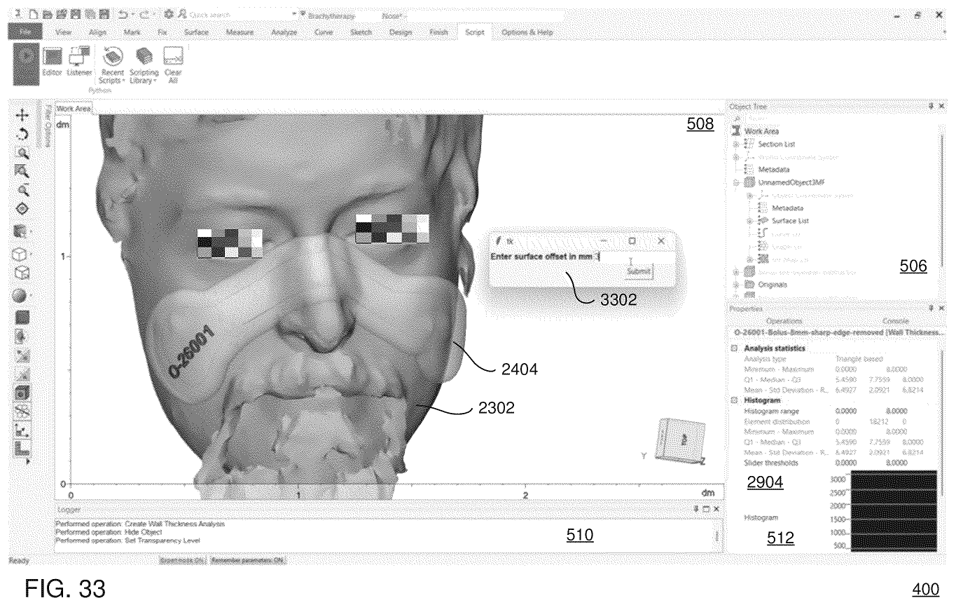

In FIG. 33, the GUI 400 shows an enter surface offset input prompt 3302, allowing the user to define an offset distance of the brachytherapy channels from the surface of the applicator 2404 (e.g., in millimeters, etc.). This ensures accurate placement of the channels relative to the patient's anatomy.

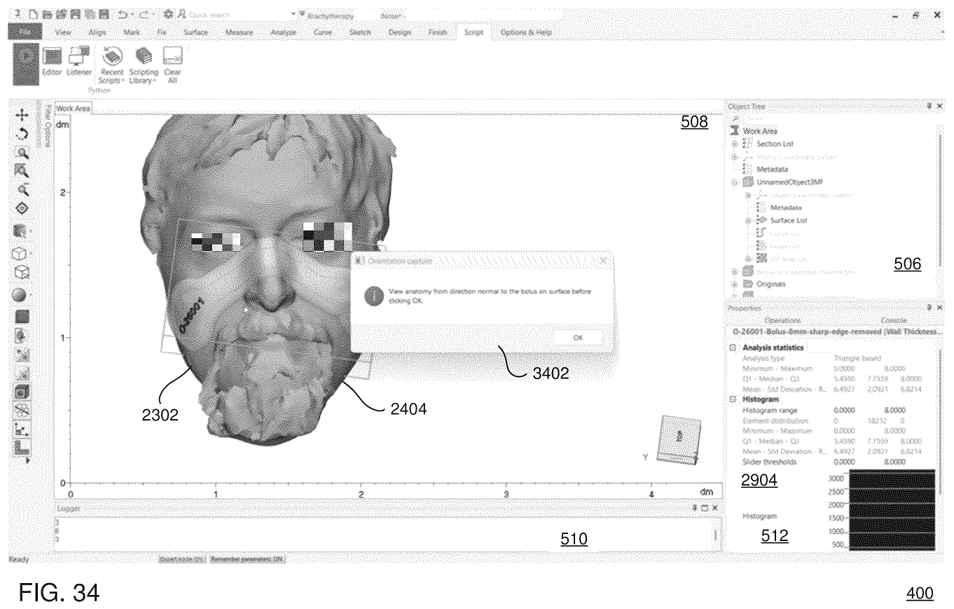

In FIG. 34, the GUI 400 displays an orientation capture prompt 3402, instructing the user to view the anatomy from a direction normal to the applicator 2404 before proceeding. This step ensures accurate alignment for brachytherapy channel placement.

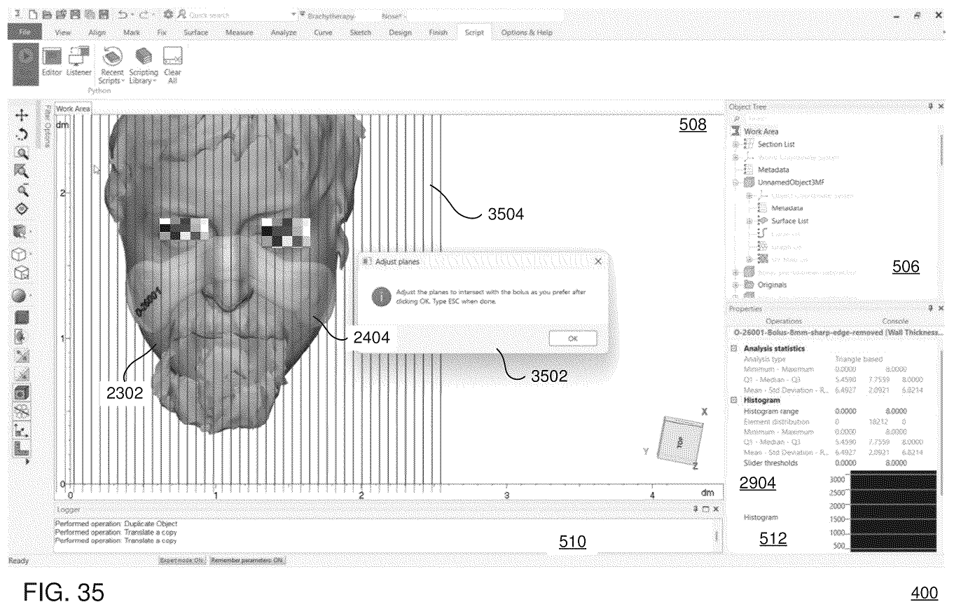

In FIG. 35, the GUI 400 presents an adjust planes prompt 3502, allowing the user to modify the intersection of alignment planes 3504 with the applicator 2404. This step ensures proper placement of brachytherapy channels.

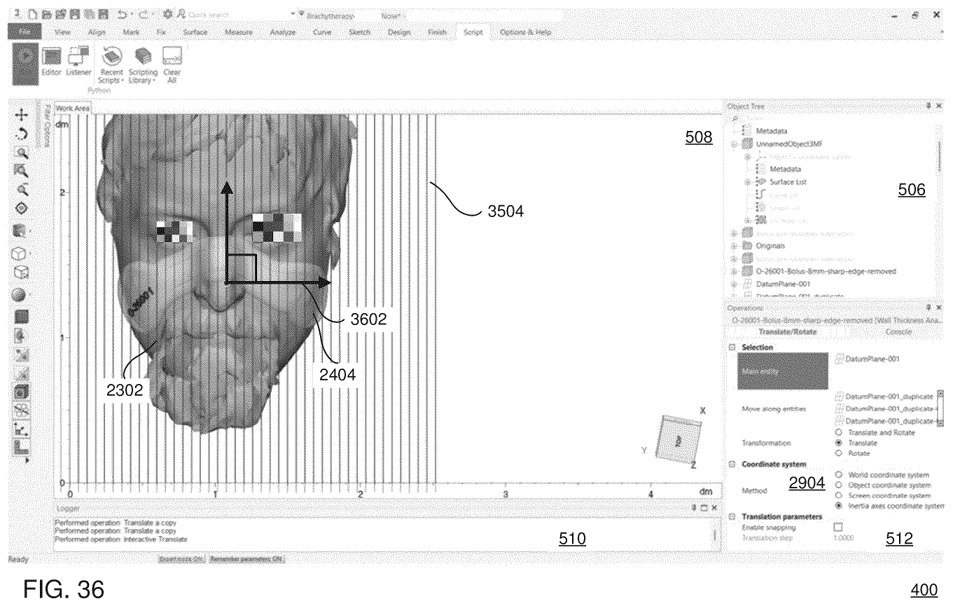

In FIG. 36, the GUI 400 presents translation controls 3602, allowing the user to adjust the position of the alignment planes 3504 relative to the applicator 2404. This step fine-tunes the placement of brachytherapy channels to ensure accurate positioning.

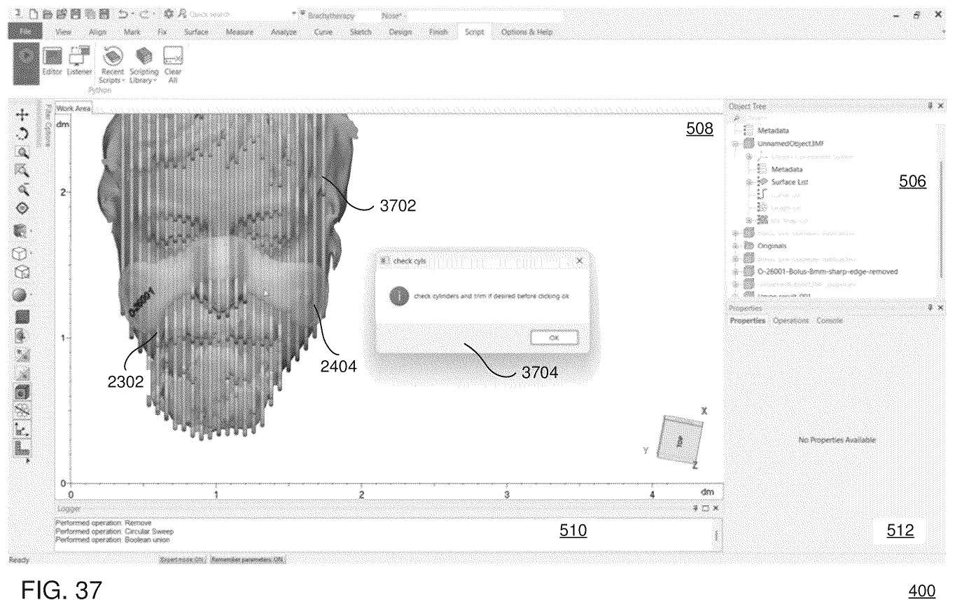

In FIG. 37, the GUI 400 displays a check cylinders and trim prompt 3704, allowing the user to review and trim cylinder-based brachytherapy channels 3702 before finalizing. These channels pass through the applicator 2404, ensuring precise catheter placement.

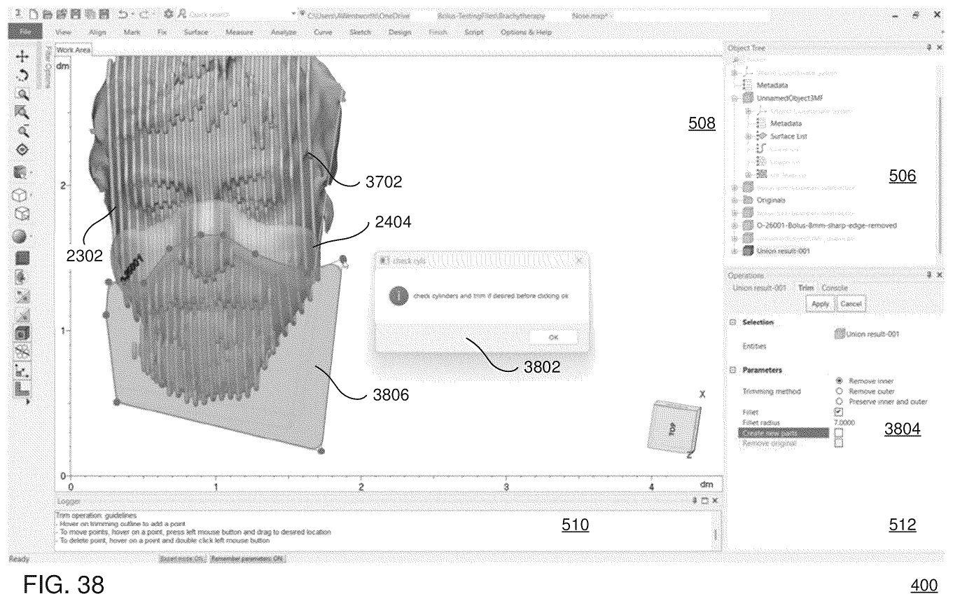

In FIG. 38, the GUI 400 presents a check cylinders prompt 3802, allowing the user to verify and trim cylinder-based brachytherapy channels 3702. A trim operation panel 3804 provides options for modifying the channels, while a trimming boundary 3806 outlines the area for adjustments within the applicator 2404.

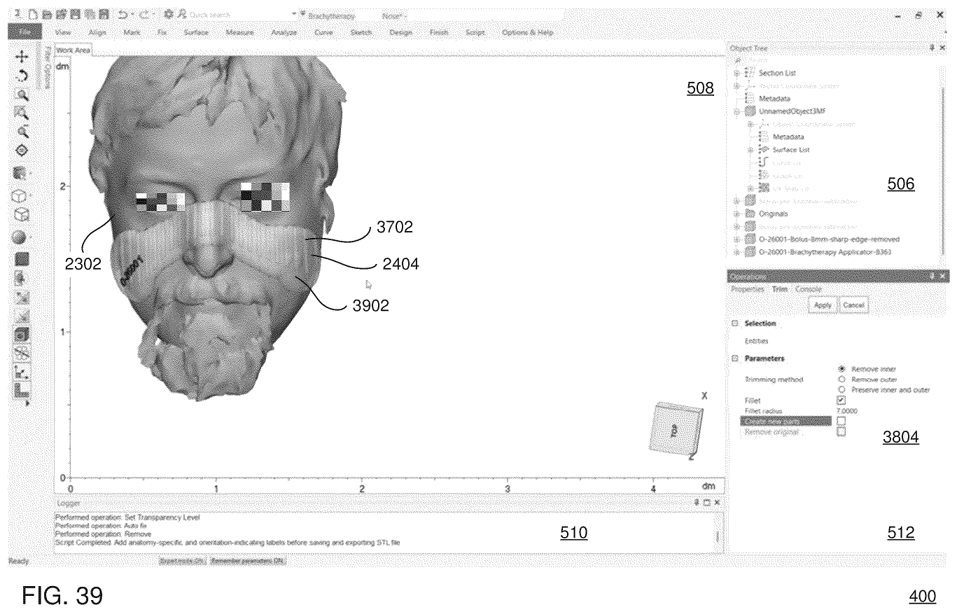

In FIG. 39, the GUI 400 displays finalized brachytherapy channels 3702 embedded within the applicator 2404, ensuring precise catheter placement. The trimmed cylindrical pathways 3902 remain visible, following adjustments made in the trimming panel 3804.

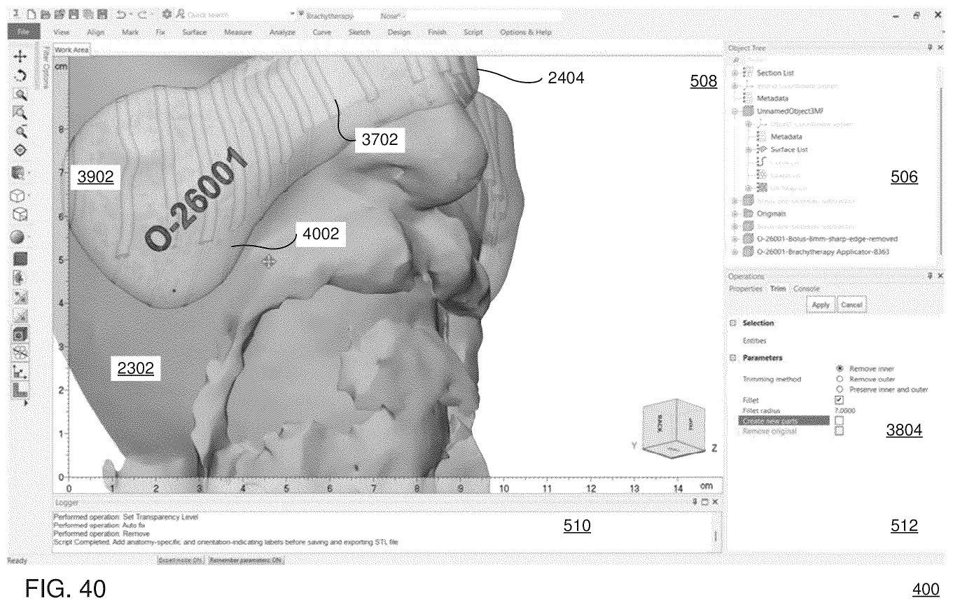

In FIG. 40, the GUI 400 displays the final brachytherapy applicator 2404 with brachytherapy channels 3702 designed for precise radiation treatment. Finer channels 4002 can be generated and that connect to brachytherapy channels 3702, preventing the formation of blind holes that could trap residual resin during SLA printing, and the like. This feature improves cleanability, as channels 4002 allow isopropyl alcohol flushing to remove uncured resin in the brachytherapy channels 3702. The patient anatomy 2302 is partially visible beneath the brachytherapy applicator 2404, demonstrating the fit and patient-specific integration thereof.

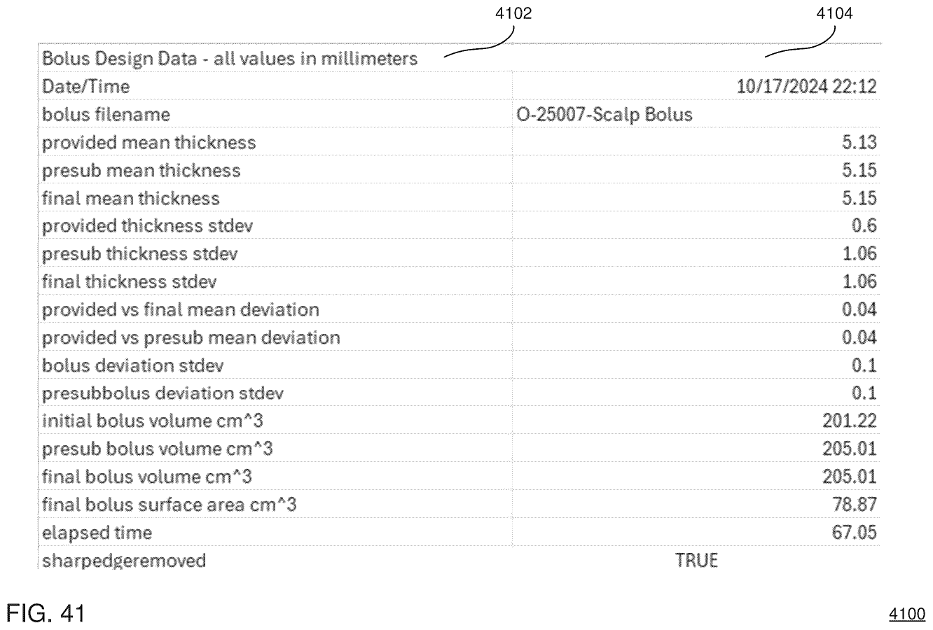

FIG. 41 shows design information generated by the bolus scripts of FIGS. 4-40, according to the present disclosure. In FIG. 41, the bolus design information 4100 for a scalp bolus includes advantageous parameters listed in column 4102, such as mean thickness values (provided, presub, and final), standard deviations (provided, presub, and final), and deviations between the provided, presub, and final stages, which help assess the accuracy and consistency of the bolus design. The data also includes volume calculations (initial, presub, and final bolus volumes) and final bolus surface area, ensuring the bolus meets the size and fit requirements. Additionally, the elapsed time for the design process is recorded, and it is confirmed whether sharp edges were removed for patient safety. The comprehensive information 4100 also contributes to quality control, as the logged measurements, deviations, and the verification of sharp edges removal ensure that the bolus complies with clinical specifications and regulatory standards. Column 4104 lists the corresponding values for parameters 4102, providing a detailed quality assurance overview, ensuring the bolus is fit for radiotherapy applications.

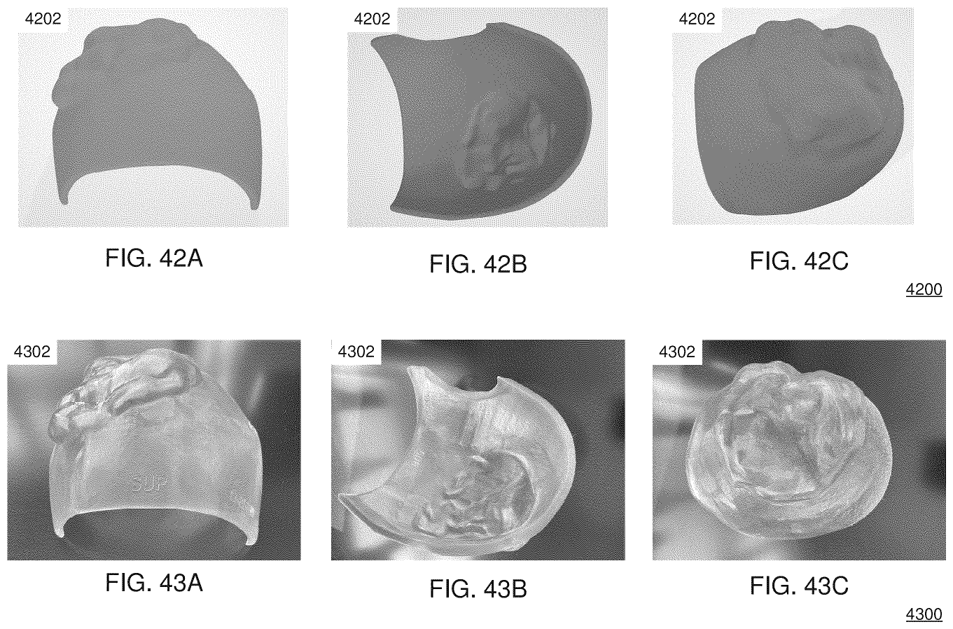

FIGS. 42A-42C show an STL file 3D render of a scalp bolus design of FIG. 41 captured from different perspectives, according to the present disclosure. In FIGS. 42A-42C, 3D renderings 4200 of a scalp bolus design from FIG. 41 are shown from three different perspectives. FIG. 42A 4202 displays a frontal view of the bolus, FIG. 42B 4204 presents a side view, and FIG. 42C 4206 shows a top view, each illustrating the detailed contours and shape of the bolus design.

FIGS. 43A-43C show a 3D print of the STL file 3D render of FIGS. 42A-42C, according to the present disclosure. In FIGS. 43A-43C, 3D prints 4300 of the scalp bolus design from FIGS. 42A-42C are shown from three different perspectives. FIG. 43A 4302 displays the printed bolus from a frontal view, FIG. 43B 4304 presents a side view, and FIG. 43C 4306 shows a top view, each highlighting the final printed form of the bolus.

Advantageously, a quality control process is employed for the bolus design of FIGS. 1-43, including thorough documentation aligned with established regulatory standards. A quality management system is employed that incorporates rigorous design validation practices to ensure compliance, for example, with ISO 13485:2016, FDA guidelines, other relevant standards, and the like. The bolus design process of FIGS. 1-43 follows strict validation protocols, ensuring the system functions as intended. The design of FIGS. 1-43 includes tests, such as mesh integrity checks, dimensional consistency, and tolerance specifications. Algorithms evaluate part properties, including volume, surface area, and geometry, to ensure compliance with allowable parameters, with thickness optimization algorithms improving constant thickness bolus designs for enhanced performance. Additionally, records related to software design, functional requirements, and design specifications are maintained in compliance with SOP-002 (Control of Records), including logging operations like mesh repairs, sharp edge removal, and Boolean operations. The bolus design process adheres to ISO 13485:2016, specifically Section 7.3 on Design and Development, and 21 CFR 820.30 on Design Controls, ensuring that the bolus meets regulatory requirements for medical device manufacturing. The FDA's General Principles of Software Validation and guidelines on Software for Medical Devices are applied to confirm that the bolus software meets validation criteria. The system generates detailed logs and quality assurance reports, including part-specific and aggregate log files, to provide traceability and ensure the design process is validated, maintaining transparency and regulatory compliance. Before export, quality inspections are performed by the system, confirming the part meets the minimum allowable volume, dimension constraints, and ensuring sharp edges are removed. The user is notified if any deviations are found, allowing corrections before finalizing the design. These quality control measures ensure the bolus design and manufacturing process of FIGS. 1-43 produces a reliable, safe, and effective product for use in medical treatments.

The above-described devices and subsystems of the illustrative embodiments can include, for example, any suitable servers, workstations, PCs, laptop computers, PDAs, Internet appliances, handheld devices, cellular telephones, wireless devices, other devices, and the like, capable of performing the processes of the illustrative embodiments. The devices and subsystems of the illustrative embodiments can communicate with each other using any suitable protocol and can be implemented using one or more programmed computer systems or devices.

One or more interface mechanisms can be used with the illustrative embodiments, including, for example, Internet access, telecommunications in any suitable form (e.g., voice, modem, and the like), wireless communications media, and the like. For example, employed communications networks or links can include one or more wireless communications networks, cellular communications networks, G3 communications networks, Public Switched Telephone Network (PSTNs), Packet Data Networks (PDNs), the Internet, intranets, a combination thereof, and the like.

It is to be understood that the devices and subsystems of the illustrative embodiments are for illustrative purposes, as many variations of the specific hardware used to implement the illustrative embodiments are possible, as will be appreciated by those skilled in the relevant art(s). For example, the functionality of one or more of the devices and subsystems of the illustrative embodiments can be implemented via one or more programmed computer systems or devices.

To implement such variations as well as other variations, a single computer system can be programmed to perform the special purpose functions of one or more of the devices and subsystems of the illustrative embodiments. On the other hand, two or more programmed computer systems or devices can be substituted for any one of the devices and subsystems of the illustrative embodiments. Accordingly, principles and advantages of distributed processing, such as redundancy, replication, and the like, also can be implemented, as desired, to increase the robustness and performance of the devices and subsystems of the illustrative embodiments.

The devices and subsystems of the illustrative embodiments can store information relating to various processes described herein. This information can be stored in one or more memories, such as a hard disk, optical disk, magneto-optical disk, RAM, and the like, of the devices and subsystems of the illustrative embodiments. One or more databases of the devices and subsystems of the illustrative embodiments can store the information used to implement the illustrative embodiments of the present disclosure. The databases can be organized using data structures (e.g., records, tables, arrays, fields, graphs, trees, lists, and the like) included in one or more memories or storage devices listed herein. The processes described with respect to the illustrative embodiments can include appropriate data structures for storing data collected and/or generated by the processes of the devices and subsystems of the illustrative embodiments in one or more databases thereof.

All or a portion of the devices and subsystems of the illustrative embodiments can be conveniently implemented using one or more general purpose computer systems, microprocessors, digital signal processors, micro-controllers, and the like, programmed according to the teachings of the illustrative embodiments of the present disclosure, as will be appreciated by those skilled in the computer and software arts. Appropriate software can be readily prepared by programmers of ordinary skill based on the teachings of the illustrative embodiments, as will be appreciated by those skilled in the software art. Further, the devices and subsystems of the illustrative embodiments can be implemented on the World Wide Web. In addition, the devices and subsystems of the illustrative embodiments can be implemented by the preparation of application-specific integrated circuits or by interconnecting an appropriate network of conventional component circuits, as will be appreciated by those skilled in the electrical art(s). Thus, the illustrative embodiments are not limited to any specific combination of hardware circuitry and/or software.

Stored on any one or on a combination of computer readable media, the illustrative embodiments of the present disclosure can include software for controlling the devices and subsystems of the illustrative embodiments, for driving the devices and subsystems of the illustrative embodiments, for enabling the devices and subsystems of the illustrative embodiments to interact with a human user, and the like. Such software can include, but is not limited to, device drivers, firmware, operating systems, development tools, applications software, and the like. Such computer readable media further can include the computer program product of an embodiment of the present disclosure for performing all or a portion (if processing is distributed) of the processing performed in implementing the disclosure. Computer code devices of the illustrative embodiments of the present disclosure can include any suitable interpretable or executable code mechanism, including but not limited to scripts, interpretable programs, dynamic link libraries (DLLs), Java classes and applets, complete executable programs, Common Object Request Broker Architecture (CORBA) objects, Artificial Intelligence (AI), Machine Learning (ML), Internet of Things (IoT), and the like. Moreover, parts of the processing of the illustrative embodiments of the present disclosure can be distributed for better performance, reliability, cost, and the like.

As stated above, the devices and subsystems of the illustrative embodiments can include computer readable medium or memories for holding instructions programmed according to the teachings of the present disclosure and for holding data structures, tables, records, and/or other data described herein. Computer readable medium can include any suitable medium that participates in providing instructions to a processor for execution. Such a medium can take many forms, including but not limited to, non-volatile media, volatile media, transmission media, and the like. Non-volatile media can include, for example, optical or magnetic disks, magneto-optical disks, and the like. Volatile media can include dynamic memories, and the like. Transmission media can include coaxial cables, copper wire, fiber optics, and the like. Transmission media also can take the form of acoustic, optical, electromagnetic waves, and the like, such as those generated during radio frequency (RF) communications, infrared (IR) data communications, and the like. Common forms of computer-readable media can include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

While the present disclosure has been described in connection with a number of illustrative embodiments, and implementations, the present disclosure is not so limited, but rather covers various modifications, and equivalent arrangements, which fall within the purview of the appended claims.

What is claimed is:

1. A computer-implemented system for ordering and manufacturing 3D-printed bolus medical devices, the system comprising:
an order processing system configured to receive order information and patient imaging data for a bolus medical device;
a bolus design system coupled to the order processing system and including bolus design script device configured to generate a patient-specific bolus based on at least one of anatomical and treatment parameters related to the patient, and generate a finalized bolus design;
a production system coupled to the bolus design system and configured to receive the finalized bolus design and perform 3D printing of a bolus medical device based on the finalized bolus design; and
a delivery module configured to deliver the 3D-printed bolus medical device to a treatment facility.

2. The system of claim 1, wherein the order processing system includes an order information processing device coupled to a digital imaging and communications in medicine (DICOM) uploading device coupled to an order confirmation device coupled to the bolus design system.

3. The system of claim 1, wherein the bolus design script device is further configured to perform at least one of part selection, dimensional evaluation, smoothing operations, Boolean subtraction, deviation analysis, and labeling.

4. The system of claim 1, wherein the bolus design script device is further configured to generate a custom 3D bolus medical device, based on an imported anatomical surface scan, and based on received user input defining at least one of bolus thickness, and anatomical region selection of the custom 3D bolus medical device.

5. The system of claim 1, wherein the bolus design script device is further configured to validate a preexisting 3D bolus medical device structure by performing at least one of dimensional analysis, Boolean subtraction of an anatomical model, smoothing operations, and sharp edge removal on the preexisting 3D bolus medical device structure.

6. The system of claim 1, wherein the bolus design script device is further configured to generate a 3D-printed applicator medical device for brachytherapy, based on user input defining at least one of catheter diameter, catheter spacing, and offset distance from a body surface.

7. A computer-implemented method for ordering and manufacturing 3D-printed bolus medical devices, the method comprising:
receiving, by an order processing system, order information and patient imaging data for a bolus medical device;
generating, by a bolus design script device of a bolus design system coupled to the order processing system, a patient-specific bolus based on at least one of anatomical and treatment parameters related to the patient;
generating, by the bolus design script device, a finalized bolus design;
performing, by a production system coupled to the bolus design system, 3D printing of a bolus medical device based on the finalized bolus design; and
delivering, by a delivery module, the 3D-printed bolus medical device to a treatment facility.

8. The method of claim 7, further comprising:
receiving, by an order information processing device of the order processing system, order information;
uploading, by a digital imaging and communications in medicine (DICOM) uploading device coupled to the order information processing device, the patient imaging data; and
confirming, by an order confirmation device coupled to the DICOM uploading device and the bolus design system, the order information and the patient imaging data before generating the patient-specific bolus.

9. The method of claim 7, further comprising:
performing, by the bolus design script device, at least one of part selection, dimensional evaluation, smoothing operations, Boolean subtraction, deviation analysis, and labeling.

10. The method of claim 7, further comprising:
generating, by the bolus design script device, a custom 3D bolus medical device based on an imported anatomical surface scan; and
receiving, by the bolus design script device, user input defining at least one of bolus thickness and anatomical region selection of the custom 3D bolus medical device.

11. The method of claim 7, further comprising:
validating, by the bolus design script device, a preexisting 3D bolus medical device structure by performing at least one of dimensional analysis, Boolean subtraction of an anatomical model, smoothing operations, and sharp edge removal on the preexisting 3D bolus medical device structure.

12. The method of claim 7, further comprising:
generating, by the bolus design script device, a 3D-printed applicator medical device for brachytherapy based on user input defining at least one of catheter diameter, catheter spacing, and offset distance from a body surface.

13. A non-transitory computer-readable medium storing instructions that, when executed by at least one processor, cause the processor to perform the steps of:
receiving, by an order processing system, order information and patient imaging data for a bolus medical device;
generating, by a bolus design script device of a bolus design system coupled to the order processing system, a patient-specific bolus based on at least one of anatomical and treatment parameters related to the patient;
generating, by the bolus design script device, a finalized bolus design;
performing, by a production system coupled to the bolus design system, 3D printing of a bolus medical device based on the finalized bolus design; and
delivering, by a delivery module, the 3D-printed bolus medical device to a treatment facility.

14. The computer-readable medium of claim 13, further comprising the steps of:
receiving, by an order information processing device of the order processing system, order information;
uploading, by a digital imaging and communications in medicine (DICOM) uploading device coupled to the order information processing device, the patient imaging data; and
confirming, by an order confirmation device coupled to the DICOM uploading device and the bolus design system, the order information and the patient imaging data before generating the patient-specific bolus.

15. The computer-readable medium of claim 13, further comprising the steps of:
performing, by the bolus design script device, at least one of part selection, dimensional evaluation, smoothing operations, Boolean subtraction, deviation analysis, and labeling.

16. The computer-readable medium of claim 13, further comprising the steps of:
generating, by the bolus design script device, a custom 3D bolus medical device based on an imported anatomical surface scan; and
receiving, by the bolus design script device, user input defining at least one of bolus thickness and anatomical region selection of the custom 3D bolus medical device.

17. The computer-readable medium of claim 13, further comprising the steps of:
validating, by the bolus design script device, a preexisting 3D bolus medical device structure by performing at least one of dimensional analysis, Boolean subtraction of an anatomical model, smoothing operations, and sharp edge removal on the preexisting 3D bolus medical device structure.

18. The computer-readable medium of claim 13, further comprising the steps of:
generating, by the bolus design script device, a 3D-printed applicator medical device for brachytherapy based on user input defining at least one of catheter diameter, catheter spacing, and offset distance from a body surface.
